(19) United States
(12) Patent Application Publication
(10) Pub. No.: US 2006/0249149 A1
(43) Pub. Date: Nov. 9, 2006

(54) Method and arrangement for the tiration of physiological measuring signals in conjunction with the observation of a patient in terms of sleep-related respiratory problems

(76) Inventors: Jörg Meier, KARNEIDPLATZ 27, 81547 MUNCHEN (DE); Stefan Schätzl, Weilheim (DE); Dieter Heidmann, Geretsried (DE); Dieter Klaus, Maulburg (DE); Caspar Graf Stauffenberg, Krailling (DE); Stefan Madaus, Krailling (DE)

Correspondence Address: GOTTLIEB RACKMAN & REISMAN PC, 270 MADISON AVENUE, 8TH FLOOR, NEW YORK, NY, 100160601, US

(21) Appl. No.: 10/549,650

(22) PCT Filed: Mar. 17, 2004
(86) PCT No.: PCT/EP04/02781

§ 371 Date: Mar. 20, 2006

(30) Foreign Application Priority Data
Mar. 17, 2003 (DE) 103-11-704.0
Jun. 13, 2003 (DE) `03-26-817.0

(51) Int. Cl. A61M 16/00; A62B 7/04; A62B 7/00

(52) U.S. Cl. 128/204.18; 128/204.21; 128/204.23; 128/204.26

(57) ABSTRACT

The invention concerns a method and an arrangement for the titration of physiological measurement signals. In particular the invention concerns a method and an arrangement for detection and evaluation of a measurement signal indicative in respect of the respiratory gas flow of a sleeping person, in conjunction with the observation of sleep-related breathing disorders. The object of the invention is that of providing solutions which make it possible to detect physiological properties which are relevant in respect of respiration during a sleeping phase, in a manner which makes it possible to assess the physiological state of the person being investigated with a high level of reliability and correctly match any therapy boundary conditions that are required. In accordance with a first aspect of the present invention that object is attained by a method of providing an evaluation result indicative in respect of the physiological state on the basis of measurement signals which are related to the respiration of a person, wherein evaluation features are generated from said measurement signals using a plurality of evaluation systems, and in the framework of a result-generation step based thereon at least one evaluation result is generated, by the evaluation features being subjected to interlinking consideration, wherein the measurement signals are detected in titration sequences which are different in respect of the respiratory gas pressure level applied to the patient and the generation of at least a part of the evaluation features or the evaluation result is effected having regard to the respective titration sequence pressure.

The invention concerns a method and an arrangement for the titration of physiological measurement signals. In particular the invention concerns a method and arrangement for detecting and evaluating a measurement signal indicative in respect of the respiratory gas flow of a sleeping person, in connection with the observation of sleep-related breathing disorders.

So-called polysomnography apparatuses are known for investigating sleep-related breathing disorders, which usually have a plurality of measurement channels for detecting measurement signals for physiological state parameters of a patient. As can be seen from patent application DE 101 64 445.0 to the present applicants, for the purpose of diagnosing a patient in regard to the respiration properties thereof during a sleep phase, it is known to detect ECG signals and blood pressure signals and to record them jointly with signals which describe the respiration activity of the patient. Those signals which describe the respiration activity of the patient can be generated by way of so-called thermistors or also by way of pneumotachographs. The detected signals can be graphically reproduced in time association with each other and evaluated in the framework of specialist examination. On the basis of specialist evaluation it is possible to establish whether any breathing disorders can be obviated by a feed of the respiratory gas at an increased pressure level. Suitable therapy parameters can also be ascertained in the framework of the specialist evaluation.

In the examination of persons with symptoms of sleep-related breathing disorders consideration of the generated measurement signals in practice under some circumstances results in different findings in particular in respect of the nature and degree of severity of obstruction-relevant properties of the respiratory tracts and the general physiological state of the patient. If a set of symptoms is inadequately evaluated the problem which arises is that boundary conditions are selected for a therapy which is possibly required, which do not adequately take account of the actual physiological requirements or which at least restrict the level of therapy comfort.

The object of the invention is to provide solutions which make it possible to detect physiological properties which are relevant in respect of respiration during a sleep phase, in a way which makes it possible to assess the physiological state of the person being investigated with a high level of reliability and to correctly match any therapy boundary conditions that are required.

In accordance with a first aspect of the present invention that object is attained in accordance with the invention by a method of providing an evaluation result which is specific in respect of the physiological state, on the basis of measurement signals which are related to the respiration of a person, wherein evaluation features are generated from said measurement signals, using a plurality of evaluation systems, and in the framework of a result-generation step based thereon at least one evaluation result is generated, by the evaluation features being subjected to interlinking consideration, wherein the measurement signals are detected in titration sequences which are different in terms of the respiratory gas pressure level applied to the patient and the generation of at least a part of the evaluation features or the evaluation result is effected having regard to the respective titration sequence pressure.

In that way it is advantageously possible for evaluation features to be ascertained in the context of a patient monitoring procedure which lasts between about 6 and 8 hours, from an amount of data which is advantageously acquired in respect of its informational significance, wherein it is possible to obtain from those evaluation features with a high level of informational significance in a standardisedly repeatable manner evaluation results which can advantageously be used for configuration or characteristic field matching of an increased-pressure artificial respiration system that is possibly required, or can also form the basis for diagnosis by a doctor and thus can contribute to standardised assessment.

The term titration sequence pressure is used to denote the static pressure of the respiratory gas, as applied to the patient. The term titration sequence is used to denote a titration portion which can be defined for example in respect of its duration, a given number of breaths or also in respect of other criteria. It is advantageously possible for the titration sequence pressure to be kept substantially constant within a titration sequence.

As an alternative thereto, or for selected sequences, it is also possible for the titration sequence pressure to be controlled within a titration sequence in accordance with a pressure control concept which for example provides for weakly alternating reference pressure settings or reference pressure settings which follow a dual-level concept. Setting or controlling the pressure within a titration sequence can be effected adaptively in accordance with selected adaptation criteria. Pressure adaptation however is preferably effected in such a way that pressure changes occurring within a titration sequence are only admissible in a band width which is smaller than the average spacing of the pressure of successive titration sequences.

The length in respect of time of the titration sequences is preferably determined by sequence length criteria. Those sequence length criteria can include both minimum lengths and also maximum lengths. It is possible to provide a plurality of sequence length criteria, on the fulfilment of which depends whether a change to a following titration sequence or a validation sequence is or is not to be effected.

For the avoidance of excessively short titration sequences, at least one minimum duration and/or minimum number of breaths is established, preferably in the form of a sequence length criterion. It is also possible to provide waiting times in each or at least in selected titration sequences, in which case it is possible, at least for given evaluation operations, to disregard the measurement signals which are detected within the waiting time, to process them with a reduced priority, or to subject them to specific verification procedures. In that way it is possible to take better account of any reactions caused by the change in pressure.

It is possible for the sequence length to be dynamically matched so that, when particular features occur, during the course of a titration sequence, it can be reduced or increased in length. In dependence on the features which occur during the course of the sequence, it is possible for the priorities of the sequence length criteria to be dynamically varied or for further sequence length criteria to be temporarily switched off (priority 0). Fulfilment checking in respect of the sequence length criteria can also include checking operations in respect of obstruction indicators.

The change from one titration sequence to the next titration sequence can also be made dependent on other onward switching criteria. Preferably however precautionary measures are taken such that a predetermined minimum number of individual titration sequences is executed.

In accordance with a particular aspect of the present invention the pressure control within a titration sequence is matched to the detection of given indicators in such a way that those indicators can be ascertained with a high level of informational significance. Preferably apnoea indicators, hypopnoea indicators and flow limitation indicators are assessed.

Actuation of the titration sequences is preferably effected in accordance with a sequence control concept. That sequence control concept preferably provides at least one period of successively rising pressure stages and a period of successively falling pressure stages.

It is also possible for the sequence control concept to be so designed that it provides a plurality of titration sequences with different titration sequence pressures, wherein in the context of actuation of those titration sequence pressures, intermediate phase pressures are actuated, in which the respiratory gas pressure level is at a level which is higher than the titration sequence pressure of a preceding titration sequence and a subsequent titration sequence. In that case the intermediate phase pressures are preferably each at the same respective pressure level, in particular preferably at an expected suitable therapy pressure level.

The sequence control concept can be such that it extends over a period including a plurality of titration sequences, and over a validation period. It is also possible for use of the sequence control concept for the respiratory gas pressure level to be limited to a period which serves for the titration operation, and for that validation period to follow that on. The duration of that period which serves for titration can be established in dependence on given, continuously generated evaluation results or also the duration can be fixedly predetermined—at least in respect of a minimum and maximum duration.

The pressure setting in the context of the validation period is advantageously based on the previously acquired evaluation results. Pressure control is preferably selected in such a way that no considerable pressure fluctuations occur within the framework of the validation period.

Within the framework of the validation period, the procedure advantageously involves effecting adaptation or plausibility checking of the evaluation results and suitability checking of a patient-specific pressure control configuration.

In accordance with a particular aspect of the present invention the evaluation features generated during the individual titration sequences are subjected to interlinking consideration, wherein the interlinking time window provided for interlinking consideration is preferably larger than that time window in which the measurement signals relevant for the evaluation features were processed, in relation to the evaluation features.

The evaluation of individual titration sequences means that it is advantageously possible to generate in particular indicative evaluation features for the respective respiratory gas pressure level and to base the properties, ascertained in that way, of the respiration within a titration sequence upon evaluation which provides for interlinking consideration of the evaluation features of a plurality of titration sequences, and upon generation of the evaluation results.

The titration concept according to the invention can furnish setting parameters, for the configuration of a pressure control device of an increased-pressure respiration unit, in particular a CPAP-unit.

In accordance with a particularly preferred embodiment of the invention physiological typification of the possibly present symptoms of the person being investigated is effected on the basis of the evaluation results generated according to the invention. On the basis of the evaluation result generated in accordance with the invention, it is possible to automatically optimise the regulating characteristics of a pressure control device provided for controlling the respiratory gas pressure, so that for example after a period of use of only one night the regulating characteristics of the pressure control system is already correctly matched to the patient. Matching can also be validated in the context of the monitoring night. The required electronic components can be integrated into a patient unit.

In accordance with a particularly preferred embodiment of the invention a configuration data set is generated on the basis of the interlinking consideration, for configuration of the respiratory gas regulation of a therapy apparatus, in particular a CPAP-apparatus. The configuration data set can be transmitted by way of an interface device or advantageously also by a mobile data carrier, in the form of a memory stick or a PCMCIA card, to the therapy CPAP-apparatus. The configuration data set can be modified if required with the interposition of an adaptation procedure in such a way that it takes particular account of certain system properties of a CPAP-apparatus which is not used for titration. It is also possible for the investigation according to the invention, with subsequent validation, to be implemented directly with a patient unit, wherein preferably the control device provided for executing the pressure control concept according to the invention can be docked to an interface device of the patient unit. It is also possible for the control device provided for executing the pressure control and measurement value acquisition concept according to the invention to be used if necessary with the interposition of a power circuit for the actuation and power feed of a conveyor blower of the patient unit. It is also possible for the patient unit or the unit provided for carrying out the investigation to be used to control the desired pressure levels by a procedure whereby a mask pressure measuring device is acted upon with varying pressures, for example by way of a mask pressure measuring hose.

In accordance with a particularly preferred embodiment of the invention the evaluation features are generated on the basis of correlation criteria, by which for example common aspects with preceding breaths, or reference breaths, are assessed. The correlation criteria can in particular also be applied to the first and/or second derivative of the detected respiratory gas flow. Generation of the features which are characteristic in respect of the respective breath can be effected using statistical methods. Interlinking consideration of the properties which are ascertained for each breath can also be effected using statistical methods.

On the basis of the evaluation features which are generated within a titration sequence for each breath or also for given successions of breaths, it is possible successively to fill up a feature array from which entries can be read according to selected interlinking criteria.

In accordance with a particularly preferred embodiment of the invention the evaluation features are generated in such a way that included among them are for example evaluation features which contain the information regarding the duration of a breath and/or for example information which is characteristic in respect of breaths which are to be deemed to be normal. On the basis of those evaluation features it is possible to determine the length in respect of time of periods with normal respiration, in the context of interlinking consideration.

In accordance with a particular aspect of the present invention a feature contribution contained in the evaluation features is determined within a time window which is smaller than an interlinking time window provided for interlinking consideration. In that way it is advantageously possible, for a respective breath, to detect typical properties in the context of high-resolution consideration of the breath, and to subject the properties of the breaths, which are ascertained in that situation, to consideration which takes account of a plurality of breaths.

The evaluation concept according to the invention can be used in regard to control of the pressure of a respiratory gas fed to the patient by means of an increased-pressure artificial respiration system. In that respect it is possible for the respiratory gas pressure to be precisely matched to the instantaneous physiological demands of the patient without the sleep pattern being adversely affected by a regulating dynamic which is subjectively perceived as being incorrectly high.

The interlinking consideration of the breath properties which are ascertained for the individual breaths can extend over a time window which views for example a predetermined number, for example 30 breaths, or an adaptively optimised number of breaths. Particularly for assessing the physiological state of the patient, for example as a basis for diagnosis by a doctor, it is also possible to carry out given interlinking operations, for sleep phase-related periods, selected time intervals or also time windows embracing the entire measurement. In accordance with a particularly preferred embodiment of the invention raw data and/or intermediate results which permit particularly reliable generation of characteristic values, in particular indices, are selected on the basis of interlinking operations.

In accordance with a particularly preferred embodiment of the invention physiological typification of the possibly present symptoms of the person being investigated is effected on the basis of interlinking consideration. On the basis of the evaluation result generated in accordance with the invention, it is possible to adaptively optimise the regulating characteristics of a pressure control device provided for controlling the respiratory gas pressure, so that for example after a period of use of several days the regulating characteristics of the pressure control system are optimally matched to the patient.

In accordance with a particularly preferred embodiment of the invention a configuration data set is generated on the basis of the interlinking consideration, for configuration of the respiratory gas regulation of a CPAP-apparatus. The configuration data set can be transmitted by way of an interface device or advantageously also by a mobile storage medium, for example in the form of a PCMCIA card, to the CPAP-apparatus. The configuration data set can be modified if required with the interposition of an adaptation procedure in such a way that it takes particular account of certain system properties of the CPAP-apparatus.

In accordance with a particularly preferred embodiment of the invention the evaluation features are generated on the basis of correlation criteria, in particular statistical evaluation systems, by which for example common aspects with preceding breaths, or preferably adaptively optimised reference criteria, for example in relation to reference breaths, are assessed. The correlation criteria can in particular also be applied to the first and/or second derivative of the detected respiratory gas flow. Generation of the features which are characteristic in respect of the respective breath can be effected using statistical methods. Interlinking consideration of the properties which are ascertained for each breath can also be effected using statistical methods.

On the basis of the evaluation features which are generated for each breath or also for given successions of breaths, it is possible successively to fill up a feature array, wherein that feature field describes at least in respect of selected evaluation features a time window which is at least as large as the smallest interlinking time window provided for interlinking consideration of the evaluation features.

In accordance with a particularly preferred embodiment of the invention the evaluation features are generated in such a way that included among them are for example evaluation features which contain the information regarding the duration of a breath and/or for example information which is characteristic in respect of breaths which are to be deemed to be normal. On the basis of those evaluation features it is possible to determine the length in respect of time of periods with normal respiration, in the context of interlinking consideration.

In addition the evaluation features are advantageously generated in such a way that they include the occurrence of any flow limitation phenomena in individual breaths, and preferably also specific information representative in respect of flow limitation. On the basis of interlinking consideration of the evaluation features detected in respect of flow-limited breaths of that kind, it is then possible to describe the time duration of given properties of respiration sequences which are at least partially flow-limited.

For periods in which no respiration activity is detected, it is also possible to generate evaluation features, on the basis of which the phase length of any apnoea sequences and/or features which are characteristic in respect of properties of that apnoea phase can be generated in the context of interlinking consideration. Those evaluation features preferably include information in regard to the nature of the apnoea phases, for example whether the apnoea phase is to be classified as central, obstructive or as a combination (hybrid apnoea phase).

In accordance with a particularly preferred embodiment of the invention evaluation features of that kind are also generated for snoring phases, for phases with Cheyne-Stokes respiration and hypoventilation phases.

The evaluation features preferably also contain details or information from which it is possible to derive the body position, the head position and preferably also the degree of neck twist of the patient. Sleep phase-indicative information can already be contained in the evaluation features.

The generated evaluation features are preferably stored with association in relation to the detected breath or having regard to the position thereof in respect of time. In other words the generated evaluation features are to be associated with a defined time window—in the case of normal respiration the respective breath.

In accordance with a particularly preferred embodiment of the invention in the context of interlinking consideration an index is generated, which classifies the flow limitation, referred to hereinafter as the flow limitation index. That flow limitation index can be determined for example on the basis of the following evaluation rule:

Calculation of the flow limitation index (FLI₍ₚ₎) specific to the titration interval:

$${FLI}_{p} \frac{A}{{t}_{interval}} = \frac{[ l ]}{[ h ]}$$

wherein:

FLIₚ=index which gives the flow limitations per pressure level

A=number of flow-limited breaths

tᵢₙₜₑᵣᵥₐₗ=duration of a titration sequence.

As an alternative thereto—or in combination therewith—it is also possible to ascertain a detail characterising the flow limitation potential, for example in the form of a parameter referred to hereinafter as the flow limitation relation (FLR).

Calculation of the flow limitation relation (FLR):

$${FLR}_{i} = \frac{{t}_{flowlimitation}}{{t}_{interval}} * 100 \% = [ \% ]$$

wherein:

FLRᵢ=proportion of the flow-limited inspiration time per effectively breathed inspiration time

t_{flow limitation}=inspiration time of the flow-limited breaths

tᵢₙₜₑᵣᵥₐₗ=total inspiration time of a time interval (in hours); the time interval involves for example a pressure stage, sleep phase and so forth

By means of the FLI and the FLR it is possible to put flow-limited respiration into various dependencies and the therapy pressure applied can be assessed.

The calculations relating to flow limitation are effected on the basis of the detected respiratory gas flow by volume:
Detection of the respiration phase, that is to say detection of the expiration and inspiration phase of respiration
ascertainment of the inspiration time
assessment of breaths in dependence on:
   respiration regularity by reverse correlation, for example stable or unstable respiration time intervals, for example 5/10/30/60/90 min pressure stages, for example 4/5/6/7 . . . mbars sleep phases, for example awake, REM, NREM 1-4 sleep quality, for example according to the nature of detected events, for example apnoea, hypopnoea, generally by the number of disturbances detected body position, for example lying on the back, lying on the side breath features, for example inspirative/expirative breath volume, max. in-/exp.-volume flow, breathing rate, various time relationships between inspiration/expiration/and/or overall breath length, breathing rate change, curvature change, index change, overall breath length change and overall breath length.

The relationships ascertained in that way can be plotted in a Table and then analysed.

| | Assessment of respiration with features | | | | | | | |
|---|---|---|---|---|---|---|---|---|
| | Respiration | Normal | Flow | | | | Cheyne-Stokes | |
| | number (total) | respiration | limitation | Apnoea | Hyponoca | Snoring | respiration | Hypoventilation |
| Assessment of | Time Interval | | | | | | | |
| respiration | 5 min | | | | | | | |
| effected in | 10 min | | | | | | | |
| dependencies | 30 min | | | | | | | |
| | 60 min | | | | | | | |
| | Pressure stages | | | | | | | |
| | 4 mbars | | | | | | | |
| | 5 mbars | | | | | | | |
| | 6 mbars | | | | | | | |
| | 7 mbars | | | | | | | |
| | 8 mbars | | | | | | | |
| | 9 mbars | | | | | | | |
| | Sleep phases | | | | | | | |
| | Waking | | | | | | | |
| | REM | | | | | | | |
| | NREM 1 | | | | | | | |
| | NREM 2 | | | | | | | |
| | NREM 3 | | | | | | | |
| | NREM 4 | | | | | | | |
| | Insp/exp breath volume | | | | | | | |
| | Overall breath length | | | | | | | |
| | Quotient T insp/exp | | | | | | | |
| | Breathing rate | | | | | | | |
| | . . . | | | | | | | |
| | . . . | | | | | | | |
| | . . . | | | | | | | |
| | Body position | | | | | | | |
| | Back | | | | | | | |
| | Side | | | | | | | |

In addition it is possible to generate a snoring index in the context of interlinking consideration. The signal used for ascertaining snoring sequences can be generated from the respiratory gas pressure detecting device, from the motor power draw and also from the respiratory gas flow signal or in particular acoustic pick-up systems.

It is further possible to generate a mouth respiration/nose respiration index in the context of interlinking consideration. It is further possible to generate a sleep time index in the context of interlinking consideration.

It is further possible to generate a sleep phase index in the context of interlinking consideration. In conjunction with respiration phase consideration it is possible to draw a distinction between inspiratory (obstruction-relevant) and expiratory (less relevant) snoring. It is further possible to generate a periodic respiration index in the context of interlinking consideration. It is further possible to generate a respiration volume index in the context of interlinking consideration.

Apart from information relating to the body position, the evaluation features can be generated preferably on the basis of a volume flow measurement of the respiratory gas flow, such measurement being referred to hereinafter as v-measurement. Measurement of the respiratory gas flow can be effected at ambient temperature or also under a definedly altered respiratory gas pressure.

At least a part of the evaluation features is preferably generated having regard to the first or second derivative of the pattern in respect of time of the respiratory gas flow.

In accordance with a further aspect of the present invention the object specified in the opening part of this specification is also attained by an apparatus for carrying out the above-described method, wherein said apparatus includes a measurement signal input device and a computing device for providing a plurality of evaluation systems, wherein evaluation features are generated from said measurement signals by the evaluation systems and at least one evaluation result is generated in the context of a result-generation step based thereon, by a procedure whereby the computing device is so configured that it subjects the evaluation features to interlinking consideration and the measurement signals are detected in titration sequences which differ in respect of the respiratory gas pressure level applied to the patient and generation at least of a part of the evaluation features or the evaluation results is effected having regard to the respective titration sequence pressure.

In the context of detecting the respiration activity of the patient on the basis of data indicative in respect of the respiratory gas volume flow, it is possible to recognise individual breaths as such. The beginning and the end of the inspiration and expiration phases of the breath can be determined for example in conjunction with an investigation in respect of the first and second derivatives of the respiratory gas flow signal and also having regard to the possible breath volume. The time duration of the breath phases, the actual volume of the breath and the respiration pattern can be determined on the basis of those evaluation results.

The instantaneous physiological state of the person being investigated can be further determined by a statistical analysis of the properties of a plurality of successive breaths. Based on extraction of features for each individual breath within the titration sequence it is possible to receive a reduction in raw data. A distinction can be drawn between obstruction-relevant snoring and obstruction-irrelevant snoring, from the statistical evaluation of the properties of a plurality of successive breaths. Typification of oscillation properties in connection with snoring events can be effected in that respect, without using a microphone device.

The occurrence of any snoring-induced oscillations can be detected on the basis of the configuration in respect of time of the respiratory gas pressure. Thus it is possible for example to extract respiratory gas pressure oscillations caused by snoring from signals generated by corresponding respiratory gas pressure sensor devices. In particular on the basis of frequency and amplitude analysis, for example fast-Fourier analysis, it is possible to classify snoring events in terms of their location of origin (soft palate, larynx . . . ).

In the context of statistical evaluation of the successive breaths it is possible, for each titration sequence, to generate a respiration index which is indicative in respect of respiration stability. That respiration index is preferably determined in accordance with the following rule:

Calculation of the respiration index (At-I) for each titration sequence:

$$At \text{-} I = \frac{A}{{t}_{interval}} = \frac{[ l ]}{[ h ]}$$

At-I=index which specifies a specific kind of respiration pattern, for example unstable, stable respiration, mouth respiration, nasal respiration per hour

A=number of given respiration patterns, for example unstable breaths

tᵢₙₜₑᵣᵥₐₗ=time of the measurement interval in hours; the time interval involves for example a pressure stage, sleep phase and so forth.

In that respect stable respiration occurs when the respiration stability index is ≧0.9 and unstable respiration is deemed to be occurring if the respiration stability index is ≦0.9.

In particular the following obstructive respiratory disturbances (OSA) can be recognised on the basis of interlinking consideration of the evaluation features:

Apnoea, hypopnoea, flow-limited respiration, stable and unstable respiration and any leakage events.

A respiration disturbance is classified at an apnoea event if a respiration stoppage is detected, the length of which exceeds a predetermined time duration of for example 10 seconds.

A hypopnoea event can be deemed to be present if, after three breaths which for example are classified as normal, at least two and a maximum of three bigger breaths are detected. The inspiratory difference volume of the breaths being considered can be adopted as a further criterion in that respect.

In the respective breath being investigated, a flow limitation can be recognised if the respiratory gas flow has given plateau zones or a plurality of maxima during the inspiration phase.

Stable respiration can be deemed to be present if the respiratory flow or the respiration frequency and the amplitude of the respiratory gas flow within a predetermined time interval can be deemed to be regular. Respiration can be deemed to be stable in particular when a respiration stability index which is defined for respiration stability is of a magnitude which is ≧ the value of 0.911. No respiratory disturbances (OSA) occur during stable respiration.

Unstable respiration can be deemed to be present if the above-mentioned respiration stability index is of a value which is smaller than 0.911 and the respiratory flow is correspondingly irregular.

Irregular respiration of that kind can be classified as a respiration disturbance, in which respect, for phases of that kind, in the case of respiratory gas pressure control, that occurs with an increased level of sensitivity.

Any high-frequency oscillations occurring in a pressure signal can be classified in connection with the respiratory flow signal as being inspiratory or expiratory snoring. The evaluation features generated in respect of the occurrence of snoring can be incorporated into the interlinking consideration provided for generation of the evaluation results.

On the basis of the configuration in respect of time of the nasally communicated respiratory gas volume flow, it is also possible to classify system states such as for example variants of respiratory gas misflows (leakage), caused for example by mask application artefacts (mask problems) or expiratory mouth respiration, and permanent oral respiration. In the case of leakage the pattern in respect of time of the nasally communicated respiratory gas volume flow exhibits a quantitative shift in relation to a reference value (for example a zero line). In the case of expiratory mouth respiration between the inspiratory volume and the expiratory volume, as at least a part of the gas change takes place orally. Further key features lie in the gradient of the inspiration/expiration flank, and the relative position in respect of time of the extreme values in the respective respiration phase.

In regard to using generation in accordance with the invention of respiration results on the basis of interlinking consideration of evaluation features within a titration sequence having regard to the associated titration sequence pressure for the control of the respiratory gas pressure, it is possible to match apparatus operating parameters such as for example the switching-over characteristics of a pressure control between different pressure regulating modes. Thus for example on the basis of the ascertained evaluation results it is possible to establish on the basis of which criteria respiratory gas pressure regulation is to be effected under standard dynamics or a higher ‘sensitive dynamics’.

The regulating performance of the pressure control device is preferably so adapted for the normal or standard dynamic mode that any recognised events or defined event chains allow an increase in pressure. In the context of a so-called sensitive mode, the respiratory gas pressure can be successively incrementally reduced, in which case the system can be adapted in such a way as to react to any events occurring at a lower respiratory gas pressure, with a higher level of regulating dynamics. A change to the sensitive mode can be effected in dependence on a plurality of criteria, in particular in dependence on whether the situation involves stable respiration (respiration stability index≧0.911). Operation of the apparatus under the above-specified regulating criteria is advantageously effected after the conclusion of the titration period in the context of the validation phase.

During the normal mode the regulating performance of the pressure control device is preferably so adapted that an increase in pressure occurs when apnoea states, hypopnoea states or also flow limitations are detected. In the case of two apnoea sequences of comparatively long time duration or for example also in the case of three apnoea phases of shorter duration it is possible for the respiratory gas pressure to be successively increased. An increase in the respiratory gas pressure can also be effected when a respiration stoppage is detected over a predetermined time duration of for example 1.2 minutes. The increase in respiratory gas pressure can be effected continuously or also stepwise, in which respect the pressure increase gradient preferably does not exceed a maximum value of 4 mbars per minute. It is possible to provide a minimum pressure limit in the range of 4 to 10 hPa and a maximum pressure limit in the range of 8 to 18 hPa. Preferably a pressure in the range of 4 to 8 hPa is provided for an algorithm starting pressure. In the case of detected hypopnoea states, there is preferably a pressure increase in comparatively small pressure stages of for example 1 mbar, in which case the number of pressure increase stages is preferably limited.

In the case of flow limitation phases, with a respiration stability index of ≧0.911, a pressure increase can be caused by pressure stages each of 1 mbar. A reduction in pressure can occur if, within a predetermined time window of for example 9 minutes, stable respiration is detected and the respiration stability index is of a value of ≧0.911. In that case a reduction in pressure of for example 2 mbars can be allowed. It is also possible, for certain time periods, to suppress a change in the respiratory gas pressure or to limit it to a comparatively narrow pressure change corridor. Implementation of a change in pressure can be prevented for example when a given combination of criteria occurs, in which inter alia respiration is classified as unstable and the respiration stability index is ≦0.911.

Operation of the respiratory gas pressure regulation in the sensitive mode has the result that an increase in pressure occurs when apnoea states occur in accordance with a predetermined time pattern. Thus it is possible for example to cause an increase in pressure by 2 mbars when either a respiration stoppage of a duration of more than 2 minutes is detected or two large (at least 25 seconds) or three smaller apnoea states (max. 25 seconds) are detected and the respiratory gas pressure in that respect is below 14 mbars. An increase in pressure by a value of 1 mbar can be caused if hypopnoea sequences occur over a time interval of at least 3 minutes.

Increases in pressure by 1 mbar in each case are caused in the sensitive mode when A out of A breaths exhibit flow limitation features and the respiration stability index is ≧0.911 or B out of C breaths exhibit flow limitation features and the respiration stability index is ≦0.911 or also C out of D breaths exhibit flow limitation features and the respiration stability index in that case is also ≦0.96.

A reduction in pressure is preferably caused in the sensitive mode when the situation involves stable respiration and the period of time for that stable respiration is at least 3 minutes and at the same time the respiration stability index is ≧0.911. In that case a reduction in pressure of initially 2 mbars can be caused. In the sensitive mode it is also preferably possible not to allow a change in pressure phase-wise and to limit the change in pressure to a preferably narrow pressure change corridor. Preferably no pressure changes are allowed in particular when the respiration is classified as being unstable and obstruction states are recognised.

A hypopnoea phase can be deemed to be present if three normal breaths are followed by at least two but a maximum of three bigger breaths. In that respect there must be an inspiratory difference volume ΔV which exceeds a predetermined limit value (for example 50% of the average breath volume).

On the basis of consideration in accordance with the invention of the signal which is indicative in respect of the respiratory gas flow, it is possible to detect respiratory disturbances which at least initially do not require a change in the respiratory gas pressure. Such respiratory disturbances can be for example: swallowing, coughing, mouth breathing, expiratory mouth breathing, arousals and talking.

Detection and evaluation of the signals indicative in respect of the respiratory gas flow, in accordance with the invention, can give information for describing and visualising the physiological state of a person, in particular in regard to an illness related to sleep-related breathing disorders. Signal detection and evaluation in accordance with the invention can be use for the configuration of artificial respiration apparatuses. Signal detection and evaluation in accordance with the invention can further be put to use for the implementation of a respiratory gas feed apparatus, in particular an increased-pressure artificial respiration apparatus with self-matching pressure regulation.

In accordance with a particular aspect of the invention at least two of the following procedures are implemented in combination:

The respiratory gas pressure is set during an investigation night, in such a way that firstly a titration phase and then a validation phase are executed.

During the titration phase pressure control is effected in accordance with a pressure control concept which is designed for the detection of respiratory gas flow signals which are as informative and correct as possible.

Pressure control during the titration phase is effected in reproducible manner in accordance with a standard defined by titration procedure criteria.

The titration procedure criteria are adapted for the detection of measurement signals which permit assessment or classification of the physiological state of the patient with a high level of statistical certainty.

The degree of statistical certainty of the ascertained assessment or classification results is ascertained.

For each breath, breath-specific features are ascertained in accordance with defined analysis procedures.

The analysis procedures take account in particular of the inspiration process, the expiration process, the transition between said processes, curve properties of the respiratory gas flow pattern within each respiration cycle, combinational considerations of features of the respiratory gas flow pattern within a breath.

The common factors of breaths are ascertained.

Differences or time changes in breath features are ascertained and taken into consideration when assessing the physiological state of the patient.

Evaluation results are generated from a multiply interlinked consideration of individual features; the results when standardised in parametric fashion describe a physiological state or physiological properties.

Pressure control during the titration phase is effected in self-regulating mode in such a way that the physiological state of the patient is ascertained with a high level of informational significance.

Pressure control during the titration phase is effected in self-regulating mode in such a way that the physiological state of the patient is ascertained with a high level of titration comfort.

Pressure control during the titration phase is effected in self-regulating mode in such a way that the physiological state of the patient is ascertained with the lowest possible time requirement.

Pressure control or pressure setting during the validation phase is effected in a self-regulating mode in such a way that the plausibility of an ascertained respiratory gas pressure regulating concept, in particular the plausibility or correctness of an ascertained CPAP-therapy pressure is checked with a high level of statistical certainty.

Intermediate evaluation results are obtained in accordance with a defined standard.

The above-mentioned standard is so adapted that it permits conversion of the intermediate evaluation results into other preferably standardised parametric patient characteristics such as for example respiratory tract elasticity, respiratory tract closure pressure, respiratory tract resistance index, maximum inspiration volume flow, maximum expiration volume flow and hyperventilation safety.

Further details and features of the invention will be apparent from the description hereinafter with reference to the drawing in which:

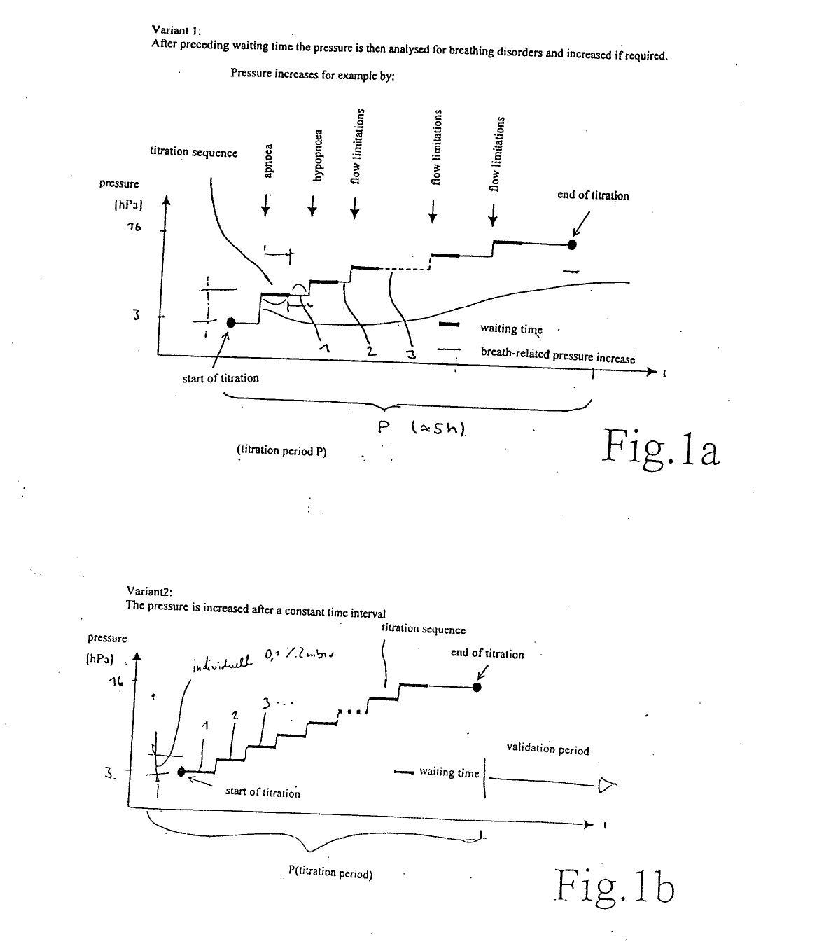

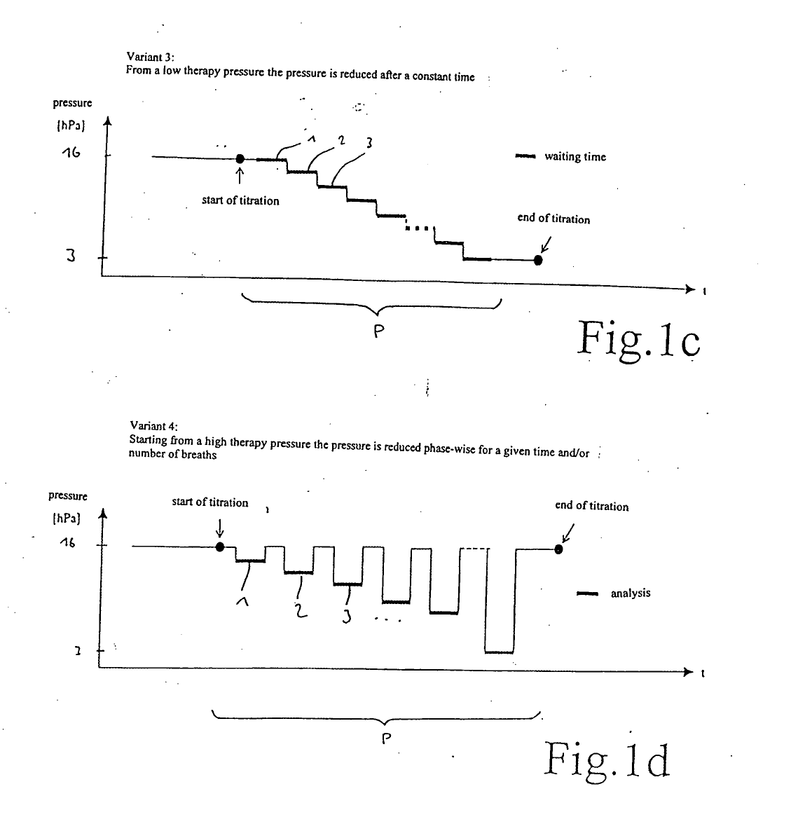

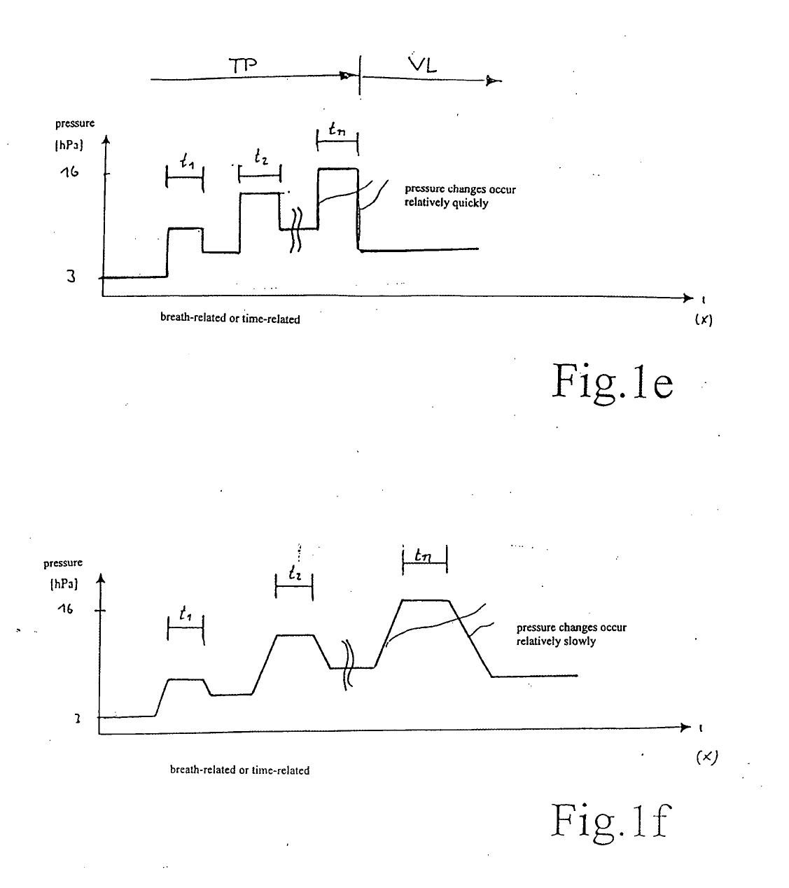

FIG. 1a shows a time chart to illustrate a titration period including a plurality of titration sequences, wherein the duration of the individual titration sequences is dynamically adapted,

FIG. 1b shows a time chart to illustrate a second variant of the titration method according to the invention with titration sequences which are established in advance in terms of their duration,

FIG. 1c shows a time chart to illustrate a portion of a titration period having a plurality of titration sequences, wherein the titration pressure is reduced stepwise from a high pressure level, the length in respect of time of the individual stages being established in accordance with a pressure control concept,

FIG. 1d shows a section from a titration period subdivided into a plurality of titration sequences,

FIG. 1e shows a time chart to illustrate a portion of a titration period with a plurality of titration sequences, wherein the titration pressure is raised stepwise from a low initial pressure level, wherein between each rise in pressure there is a temporary fall in pressure to a pressure level which is between the initial pressure level of the preceding pressure stage and the target pressure of the preceding pressure stage,

FIG. 1f shows a time chart to illustrate a portion of a titration period with a plurality of titration sequences, wherein the titration pressure is raised stepwise from a low initial pressure level, wherein between each rise in pressure there is a temporary fall in pressure to a pressure level which is between the initial pressure level of the preceding pressure stage and the target pressure of the preceding pressure stage, wherein the pressure change takes place over a period which is more extensive in comparison with the pressure control concept shown in FIG. 1e,

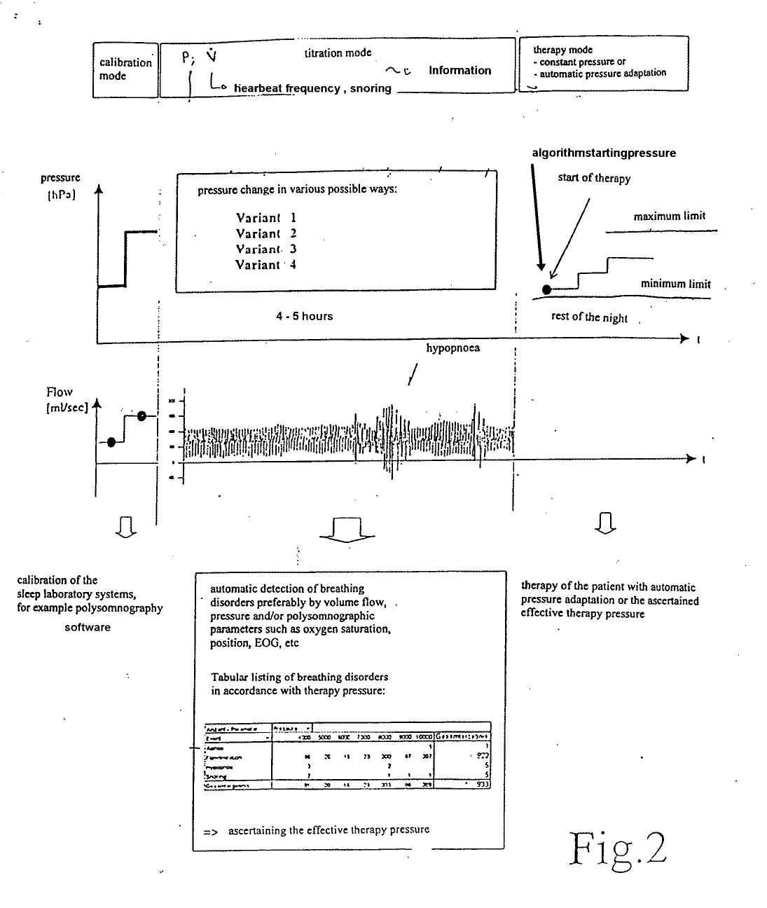

FIG. 2 shows an overview to illustrate the pressure control in a calibration mode, in the titration mode and in the validation mode,

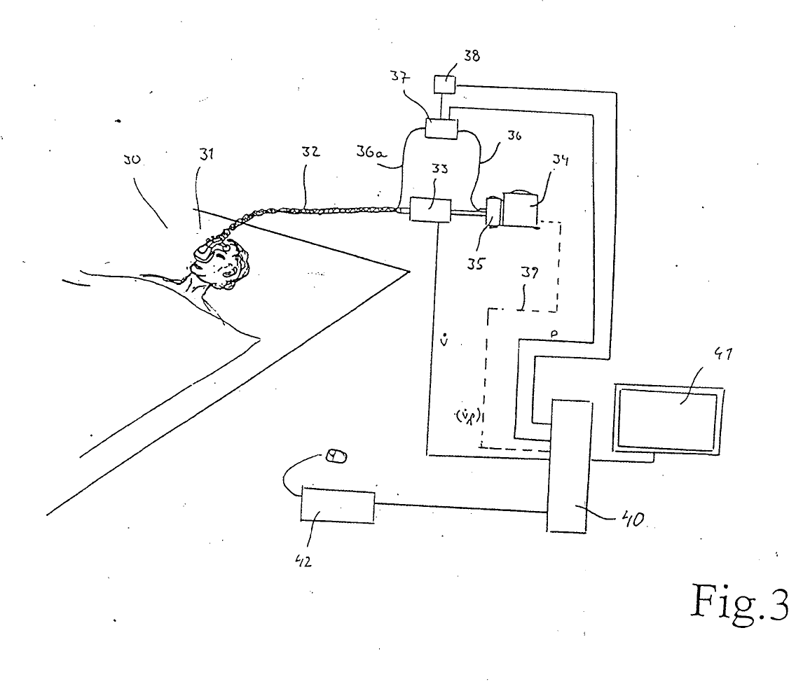

FIG. 3 shows a sketch to illustrate an arrangement according to the invention for signal titration according to the invention,

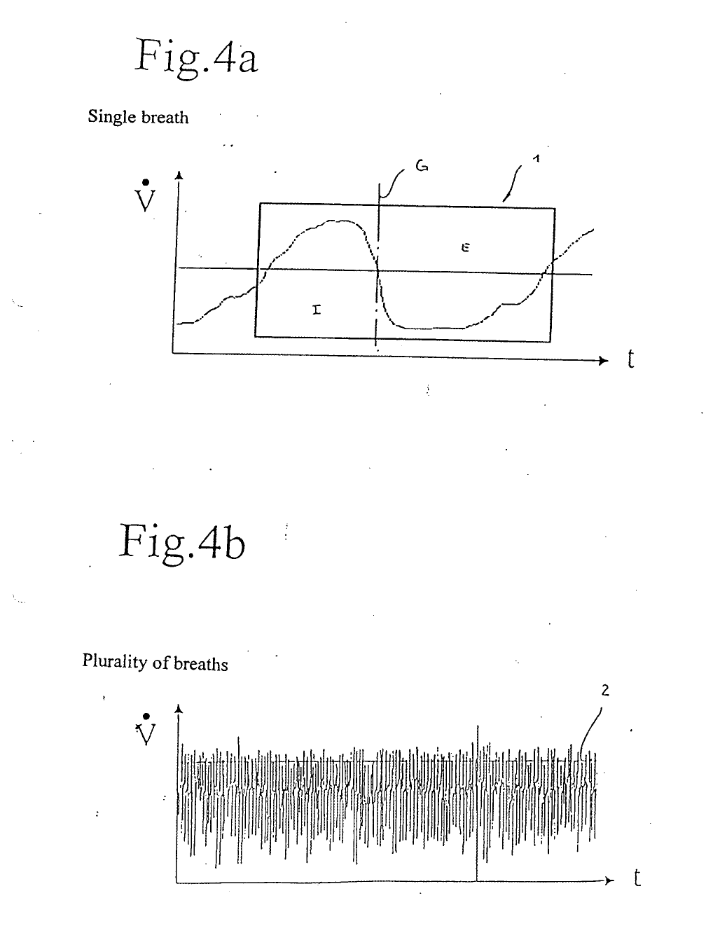

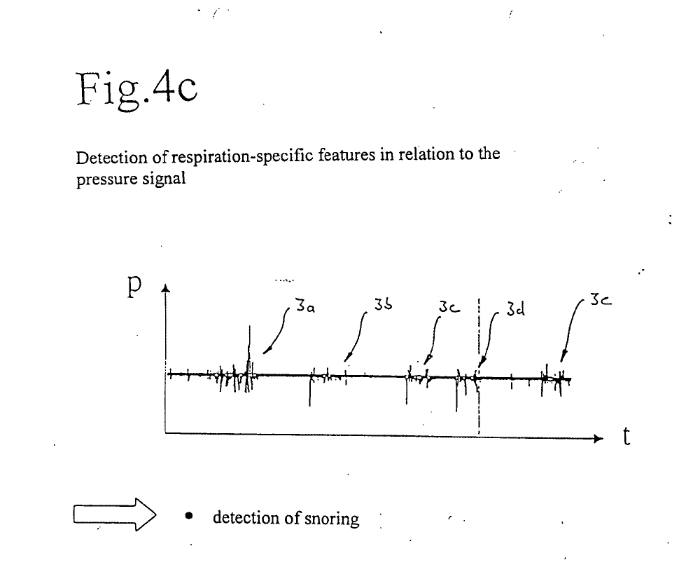

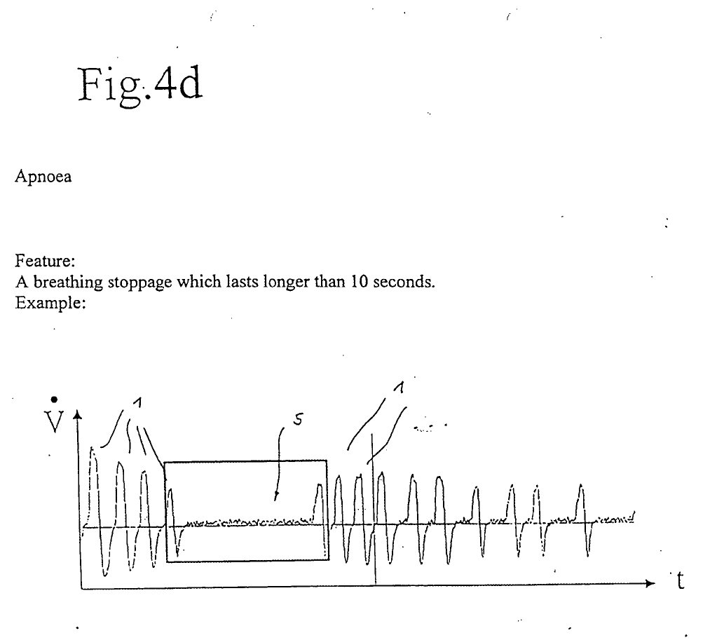

FIG. 4a shows a diagram to describe the respiratory gas flow for an individual breath,

FIG. 4b shows a diagram which describes the pattern in respect of time of the respiratory gas flow for a plurality of breaths,

FIG. 4c shows a diagram which represents the pattern in respect of time of the respiratory gas pressure with individual pressure oscillations caused by snoring,

FIG. 4d shows a diagram which represents the pattern in respect of time of the respiratory gas flow for a plurality of breaths interrupted by an apnoea period,

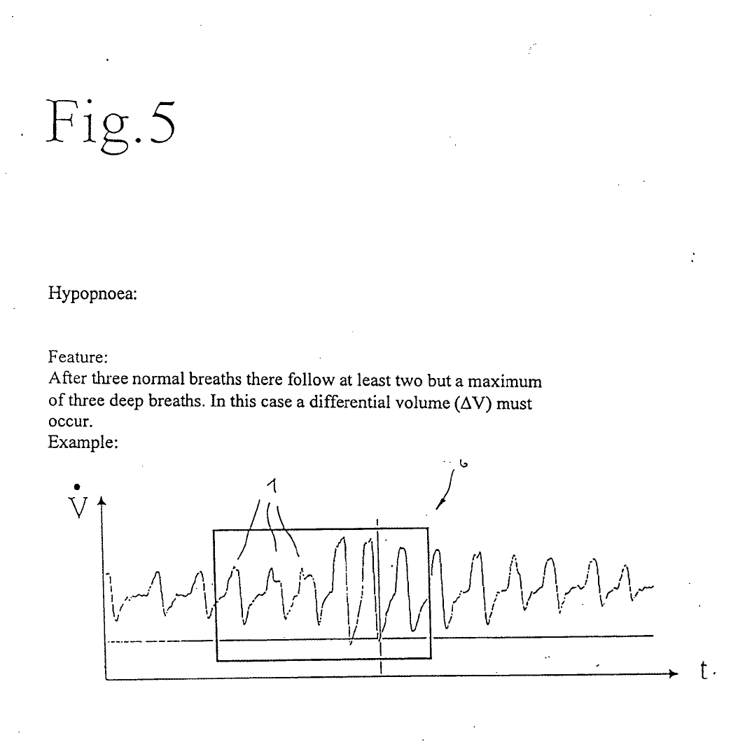

FIG. 5 shows a diagram which represents the pattern in respect of time of the respiratory gas flow with a hypopnoea event,

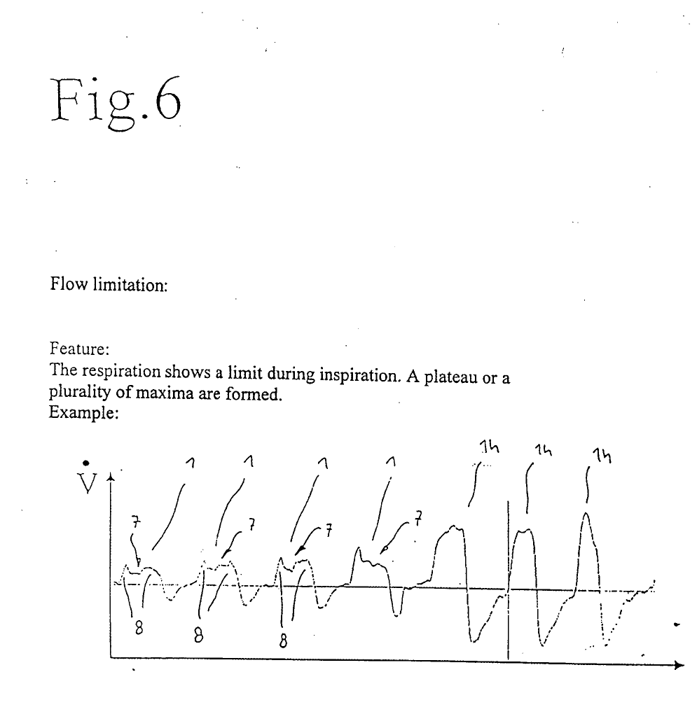

FIG. 6 shows a diagram which represents the pattern in respect of time of the respiratory gas flow for a plurality of breaths which in part are flow-limited,

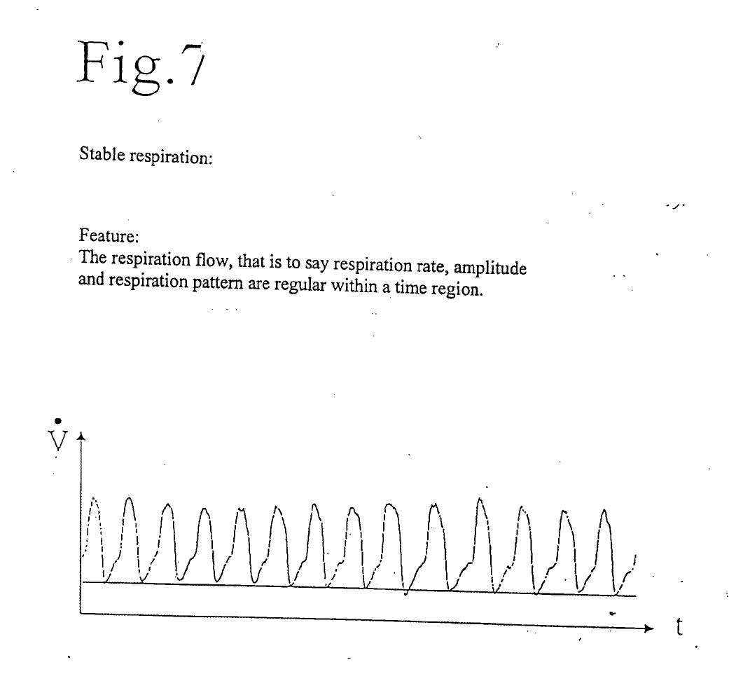

FIG. 7 shows a diagram to illustrate the pattern in respect of time of the respiratory gas flow in the case of a substantially undisturbed stable respiration,

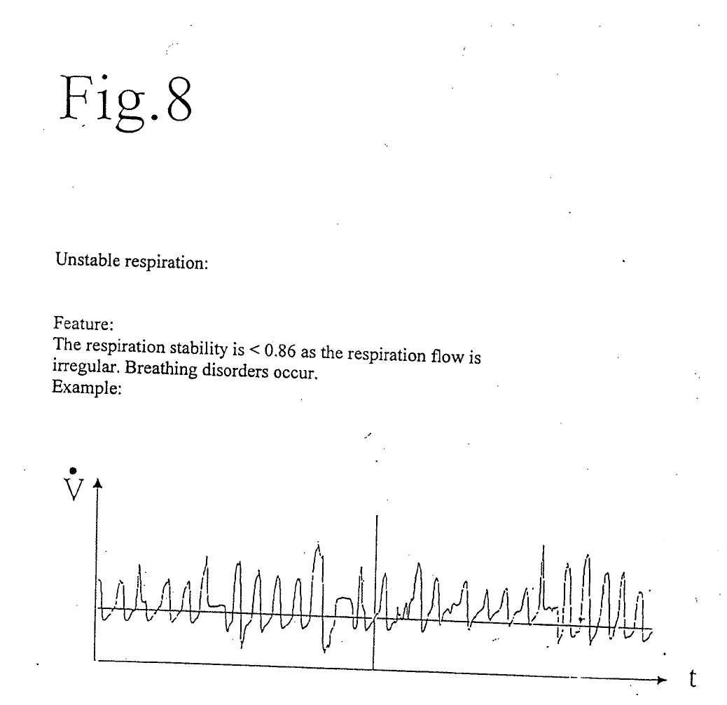

FIG. 8 shows a diagram to illustrate the pattern in respect of time of the respiratory gas flow in the case of an unstable disturbed respiration,

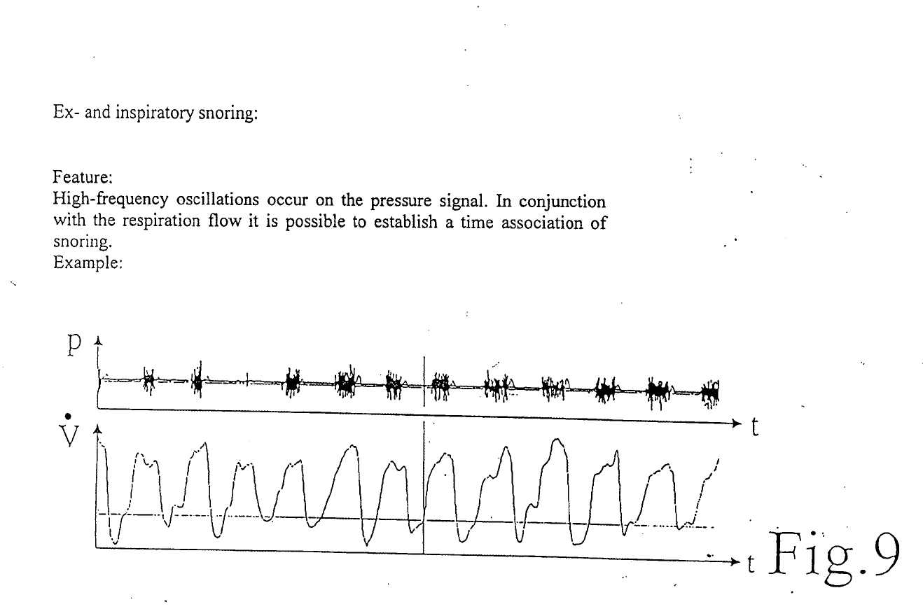

FIG. 9 shows a diagram which represents the pattern in respect of time of the respiratory gas flow and in the same time association therewith the pattern of the respiratory gas pressure wherein pressure signal oscillations caused by snoring occur therein,

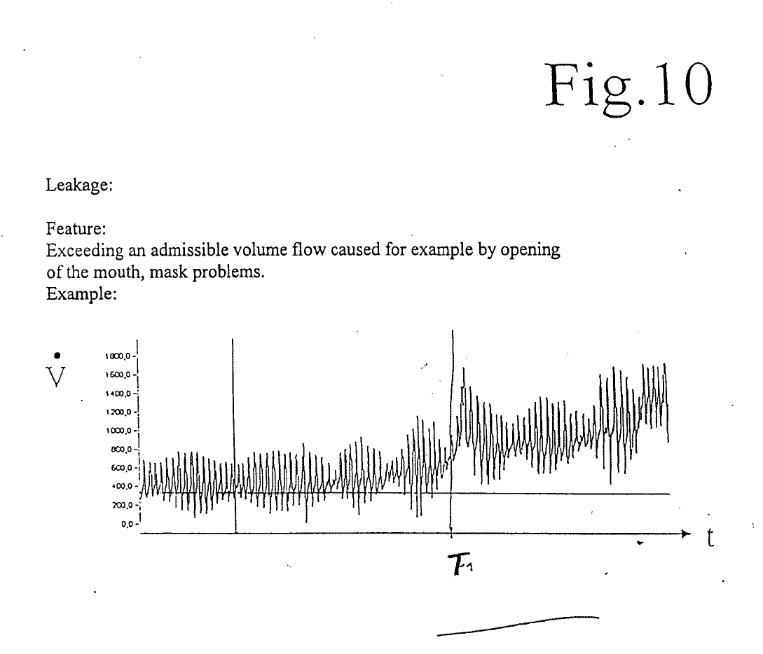

FIG. 10 shows a diagram which represents the pattern in respect of time of the respiratory gas flow in the case of a system disturbance which is caused for example by mouth breathing or mask leakage,

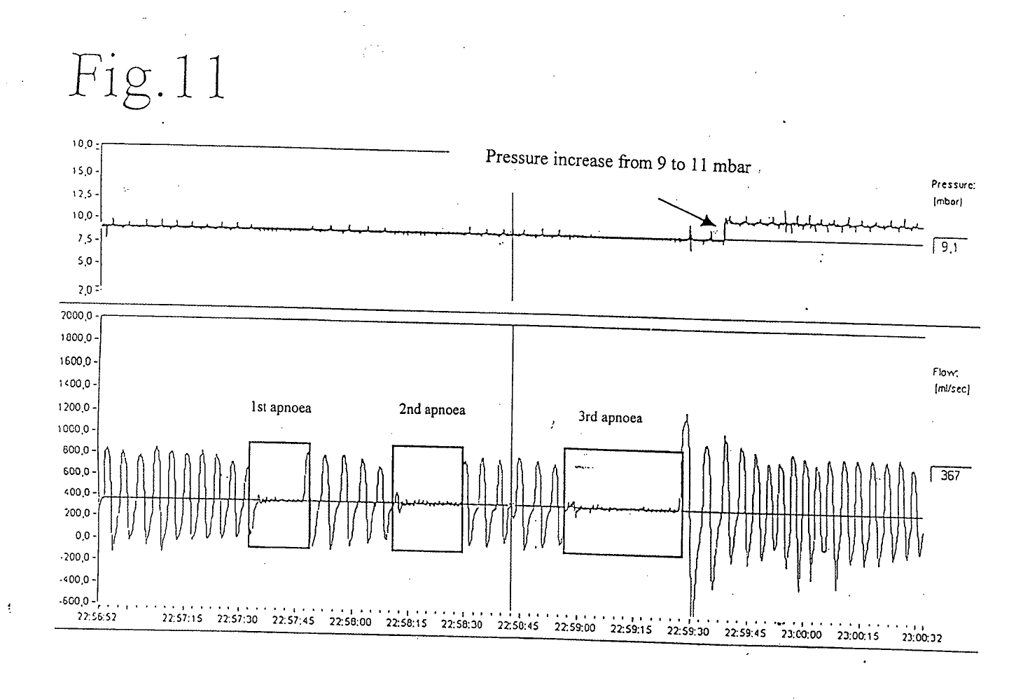

FIG. 11 shows a diagram to explain a respiratory gas pressure change caused in conjunction with the detection and interlinked consideration of respiratory patterns,

FIG. 12 shows a diagram to explain the pattern in respect of time of the respiratory gas flow and a change, implemented on the basis thereof, in the respiratory gas pressure,

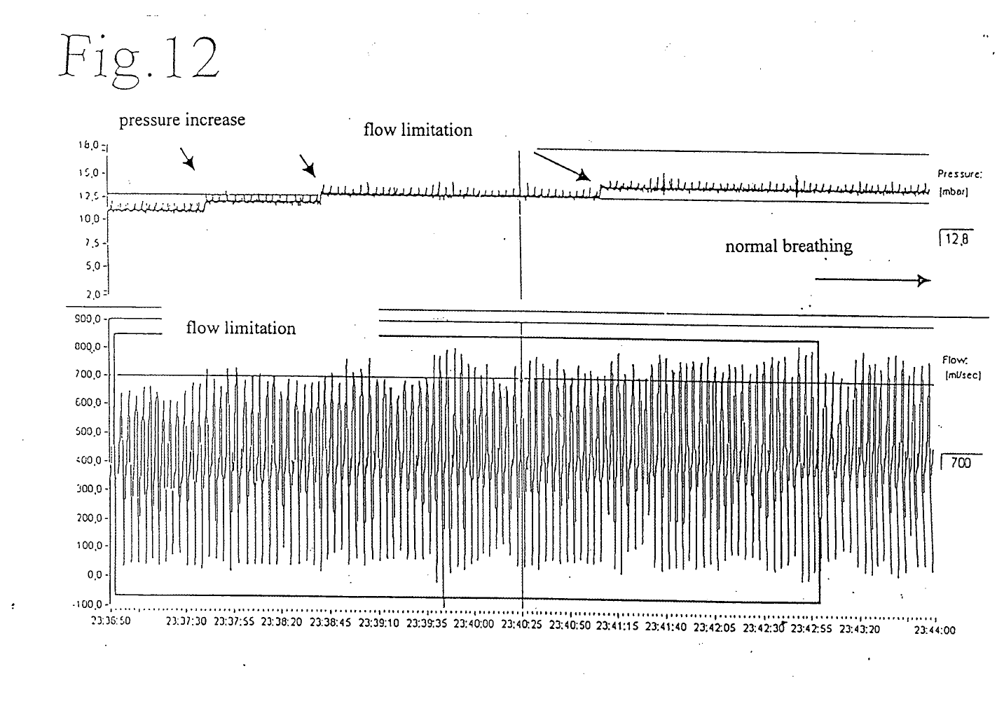

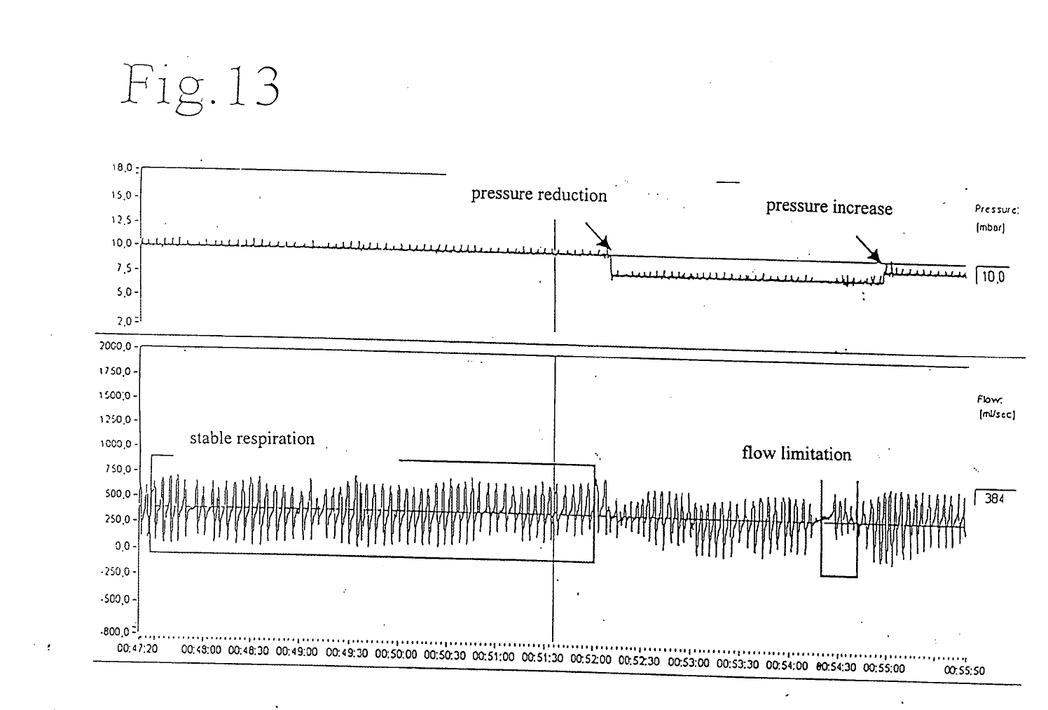

FIG. 13 shows a diagram to explain the pattern in respect of time of the respiratory gas flow in conjunction with a respiratory gas pressure change caused on the basis of said respiratory gas flow,

FIG. 14 shows a diagram to explain the pattern in respect of time of the respiratory gas flow in conjunction with a change in the respiratory gas pressure, which is caused on the basis thereof,

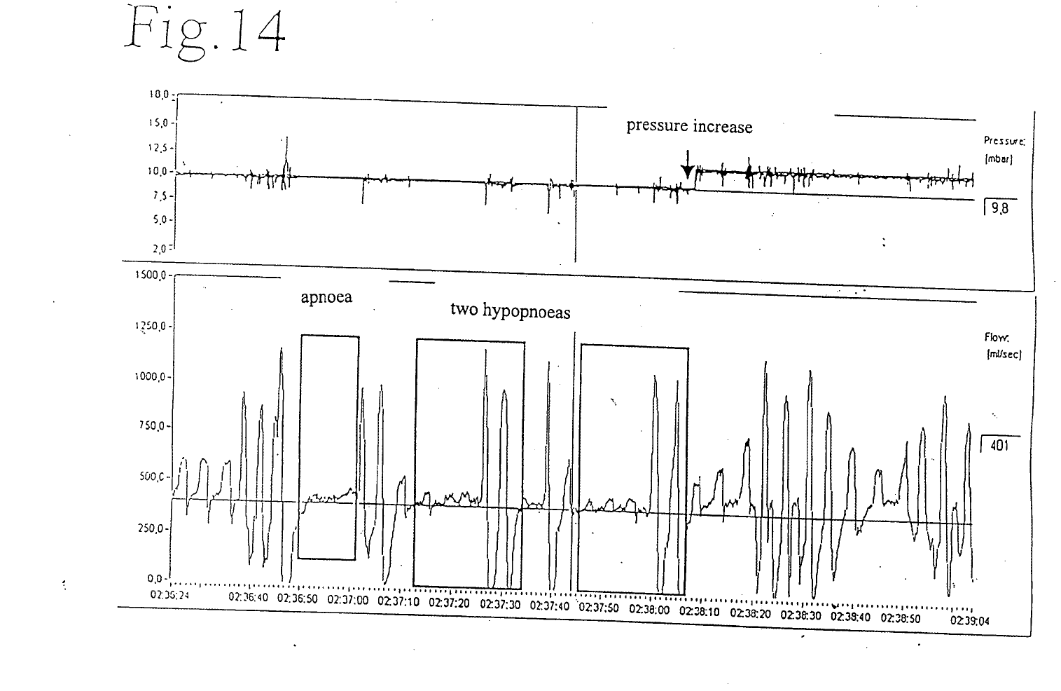

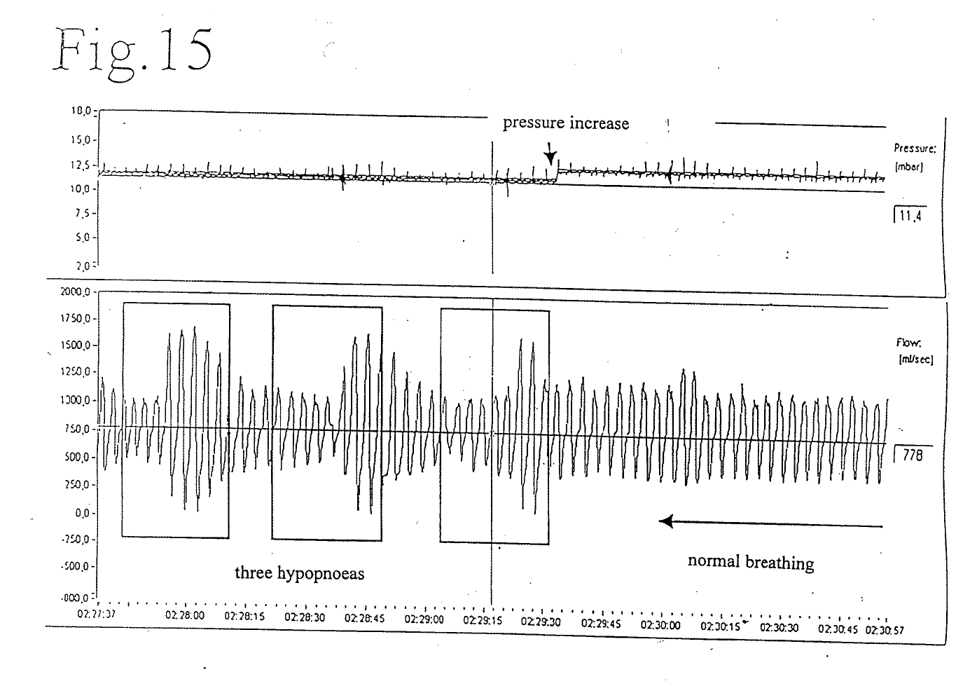

FIG. 15 shows a diagram to explain the pattern in respect of time of the respiratory gas flow with hypopnoea sequences detected therein and a respiratory gas pressure change caused on the basis of detection of said

hypopnoea sequences,

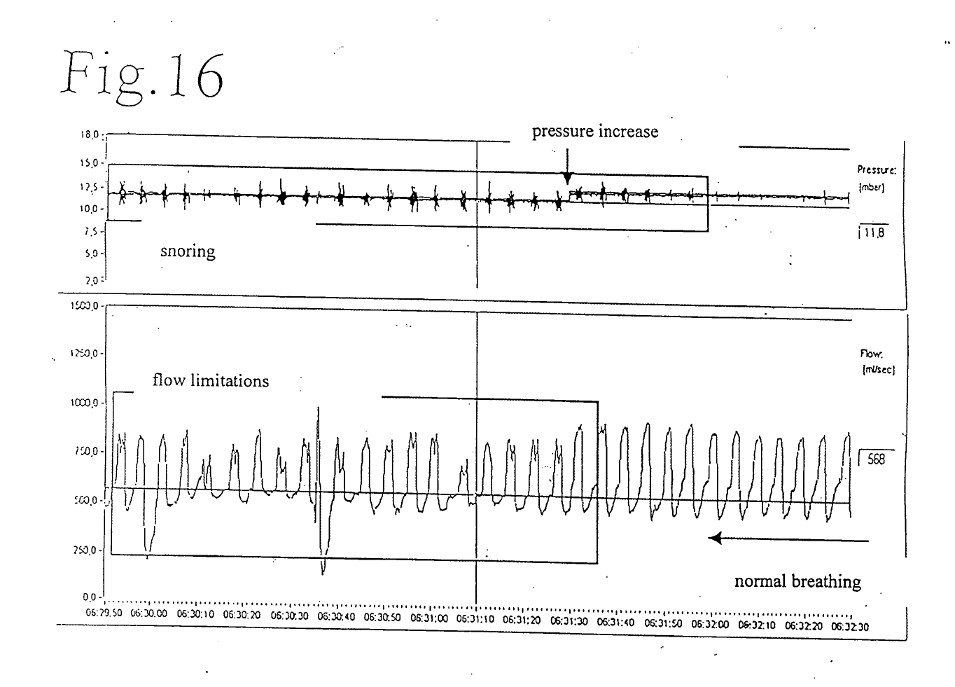

FIG. 16 shows a diagram to illustrate the pattern in respect of time of the respiratory gas flow with flow-limited breaths occurring therein and a graph to explain the respiratory gas pressure which is set in that respect,

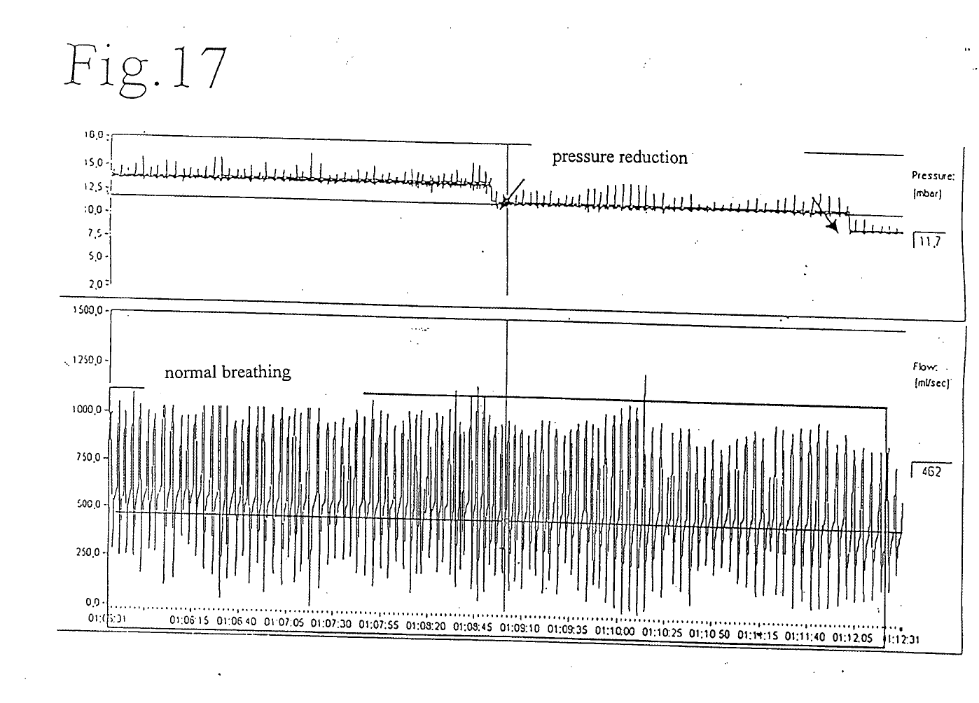

FIG. 17 shows a graph to explain the pattern in respect of time of the respiratory gas flow in conjunction with the respiratory gas pressure which obtains in this case,

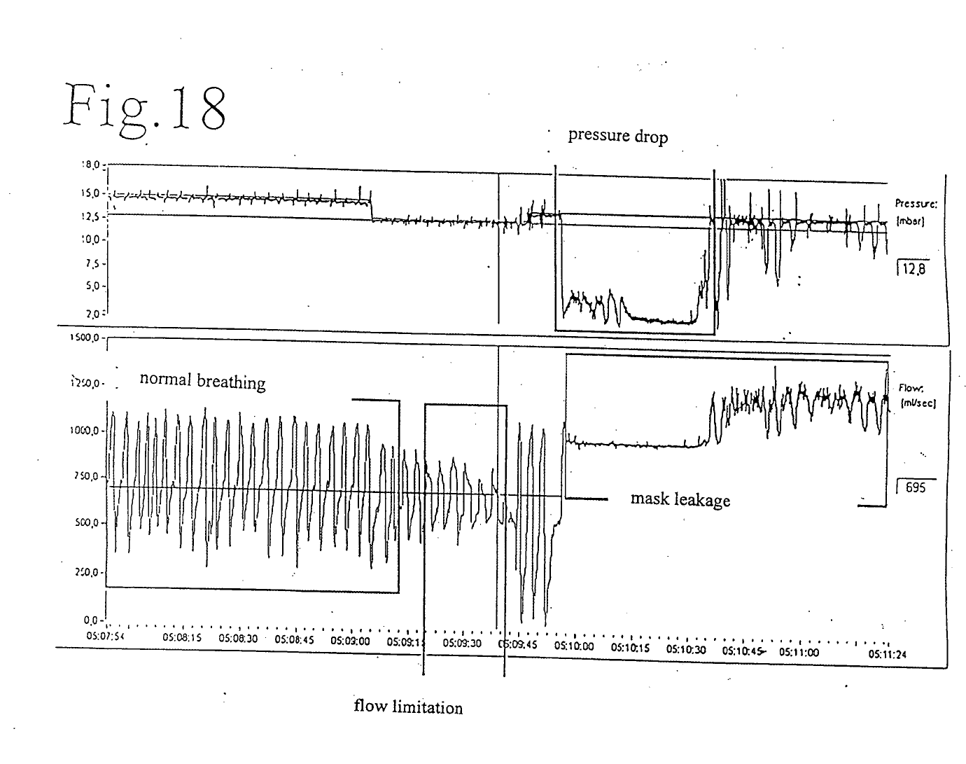

FIG. 18 shows a diagram to explain the pattern in respect of time of the respiratory gas flow with a phase occurring therein of normal respiration, a phase of flow-limited respiration, a subsequent hypopnoea phase and a disturbed phase caused by mask leakage, in conjunction with the respiratory gas pressure which obtains in that case,

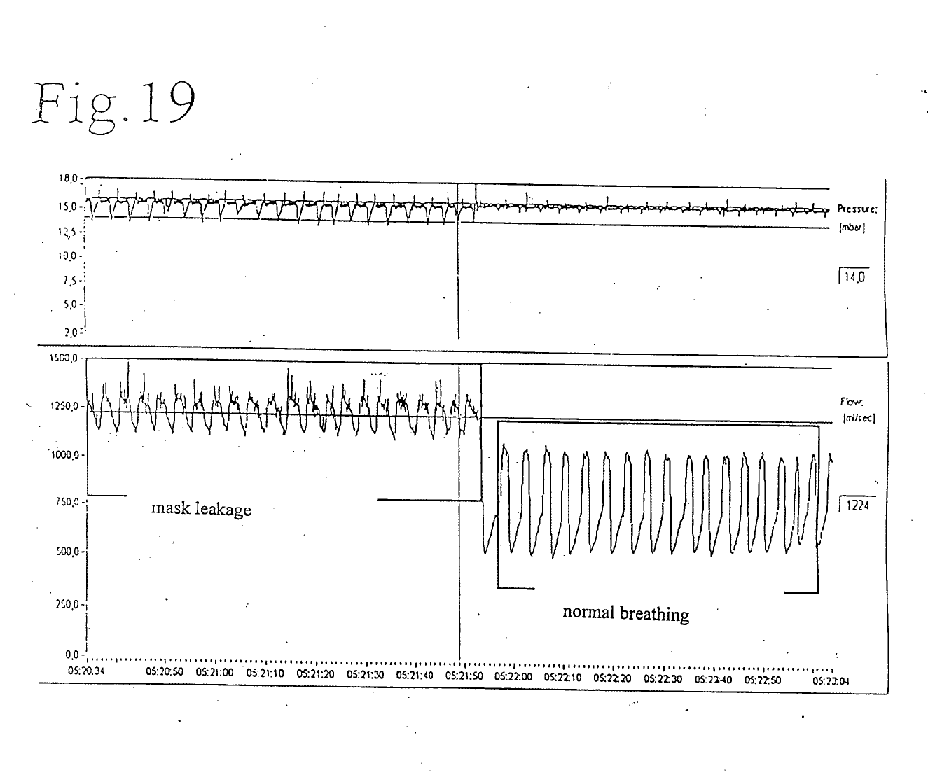

FIG. 19 shows a diagram to explain the pattern in respect of time of the respiratory gas flow in conjunction with the respiratory gas pressure which obtains in that case,

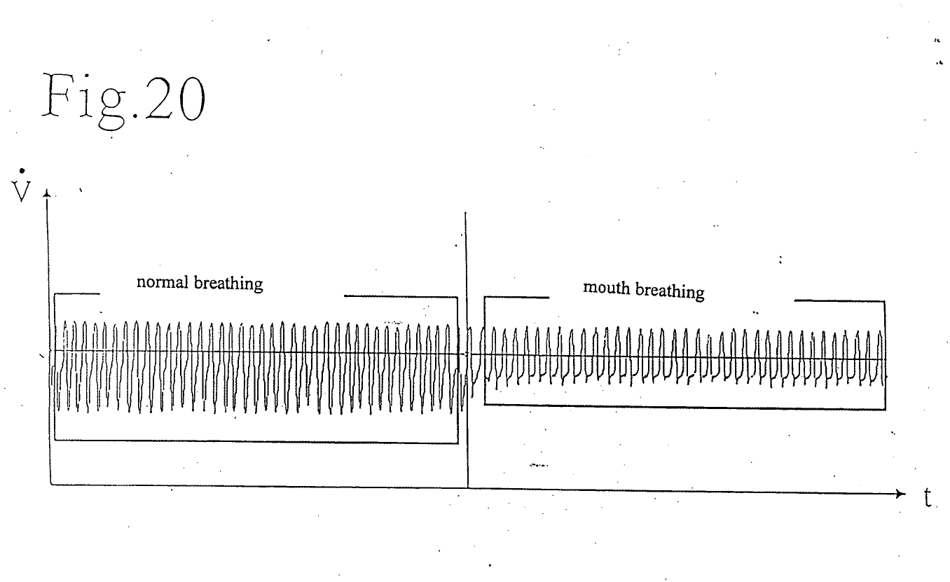

FIG. 20 shows a diagram to explain the pattern in respect of time of the respiratory gas flow for a normal respiration sequence and a subsequent sequence with additional mouth breathing, and

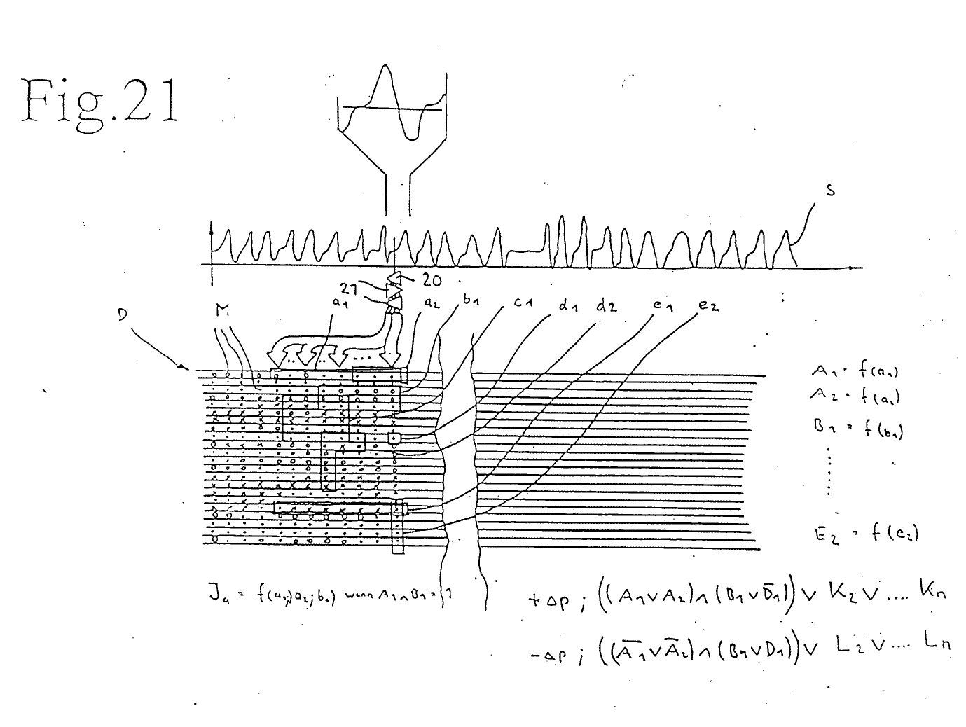

FIG. 21 shows a diagram to explain generation of the evaluation result which is specific in respect of the physiological state of a patient, on the basis of measurement signals which are in a relationship with the respiration of the person, wherein evaluation features are generated from said measurement signals, using a plurality of evaluation systems, and at least one evaluation result is generated in the context of a result-generation step based thereon, by the evaluation features being subjected to interlinked consideration.

FIG. 1a is a greatly simplified view showing the pressure, which is altered over successive following titration sequences 1, 2, 3, of the respiratory gas applied to a pressure by way of a breathing mask arrangement. In this example the set respiratory gas pressures can be in a range extending from 3 mbar to 16 mbar. The total duration of the titration period P which is here subdivided into the titration sequences 1, 2, 3 is 5 hours in this embodiment.

Due to successive actuation of titration sequences which differ in respect of the respiratory gas pressure level applied to the patient, it becomes possible to extract evaluation features from the respiratory gas flow signals and to generate from those evaluation features in the context of interlinking consideration evaluation results which for example make it possible to establish an effective CPAP-pressure or which can contribute to the typification of a set of symptoms which are possibly present.

In this variant, the pressure control concept which is crucial for pressure control in the illustrated embodiment provides that each individual titration sequence includes a waiting sequence and the respiratory gas flow or respiratory gas pressure signal is taken into consideration only after the expiry of that waiting sequence. In this embodiment, the duration of the individual titration sequences is established by boundary values, wherein within those boundary values a transition to a subsequent titration sequence can also take place when predetermined onward-switching criteria are satisfied. Those criteria involve in particular criteria which afford information as to whether the instantaneous respiration can be classified as disturbed. If the instantaneous respiration can be classified as disturbed, then in dependence on detected disturbance features it is possible to establish the residual duration of the instantaneous titration sequence and/or the jump in pressure into the next titration sequence. In particular apnoea events, hypopnoea events and flow limitation events can be used as pressure-increasing breathing disorders. The minimum duration of the individual titration sequences, which is preferably established by the waiting time, makes it possible to avoid an unacceptably rapid increase in the respiratory gas pressure. It is possible for the respiratory gas flow of the patient also to be evaluated during the waiting time periods contained in the individual titration sequences. The evaluation features which are ascertained by evaluation of the respiratory gas flow within the waiting time can form the basis for further signal processing in particular if the respiration involved satisfies predetermined criteria after the conclusion of the waiting time.

FIG. 1b shows the pattern in respect of time of the respiratory gas pressure within a titration period P, wherein in this case, as in the embodiment shown in FIG. 1, the respiratory gas pressure is increased stepwise over successive titration sequences 1, 2, 3, . . . . The pressure range in this embodiment also extends from a minimum pressure of about 3 mbar to a maximum pressure of 16 mbar. In the embodiment illustrated here the duration of each individual titration sequence is established rigidly and independently of the instantaneous respiratory gas flow. In other words, the pressure is successively increased in preferably constant intervals of time.

FIG. 1c also shows in the form of a time/pressure chart the respiratory gas pressure which is varied in accordance with a further variant of the pressure control concept during a titration period. In accordance with the pressure control concept illustrated here the respiratory gas pressure applied to the patient is set at the beginning of the titration period to a plausible maximum pressure of for example 16 mbar. Over a number of successive titration sequences 1, 2, 3 the respiratory gas pressure is successively reduced to a level of 3 mbar to the end of the titration period P.

FIG. 1d shows the pattern in respect of time of the respiratory gas pressure in accordance with a fourth variant of a pressure control concept according to the invention for a titration period P. In accordance with the pressure control concept executed here the procedure involves a reduction in the respiratory gas pressure from a high respiratory gas pressure level to a predetermined titration pressure, wherein between each of the individual titration sequences 1, 2, 3 there is a return to the increased initial pressure level, for a respective predetermined period of time. The duration of the titration period P illustrated here can be for example between 3 and 5 hours. The length in time of the individual titration sequences is preferably about 18-32 minutes. The change in the respiratory gas pressure between the following titration sequences or the return to an intermediate pressure level can take place gradually extending over a plurality of breaths. It is possible for the change in the respiratory gas pressure to be effected in such a way that in particular increases in pressure occur only in given respiration phases, for example during the expiration phases.

FIG. 1e shows a time chart to illustrate a portion of a titration period comprising a plurality of titration sequences, wherein the titration pressure is raised stepwise from a low initial pressure level, wherein between each increase in pressure there is a temporary drop in pressure to a pressure level which is between the initial pressure level of the preceding pressure stage and the target pressure of the preceding pressure stage. The individual titration sequences t1, t2 . . . tn can be fixed in respect of their duration, the number of breaths to be investigated or other titration sequence length criteria. The pressure changes which occur between the individual pressure stages take place relatively quickly, preferably within the transition from the inspiration to the expiration phase. After termination of the titration phase TP having pressure stages, a validation phase VL is implemented, in which a respiratory gas pressure is set, which is established on the basis of evaluation results which were ascertained during the titration phase, and is evaluated in respect of its plausibility by further assessment features.

FIG. 1f shows a time chart to illustrate a portion of a titration period with a plurality of titration sequences, wherein the titration pressure is raised stepwise from a low initial pressure level, wherein between each rise in pressure there is a temporary reduction in pressure to a pressure level which is between the initial pressure level of the preceding pressure stage and the target pressure of the preceding pressure stage; and wherein the change in pressure takes place over a period of time which is extended in comparison with the pressure control concept shown in FIG. 1e. The respective change in pressure is preferably effected over between about 10 and 15 breaths. The further description relating to FIG. 1e applies in a corresponding fashion here.

FIG. 2, divided into four levels, shows details relating to a calibration mode, illustrating the titration mode described hereinbefore in four alternative forms, and a therapy mode which is set forth in accordance with the invention hereinafter.

During a calibration mode which is executed prior to the titration mode, calibration of the measuring arrangements and in particular the sleep laboratory systems, and basic configuring of an electronic evaluation system can be implemented. That calibration mode can extend over a period of for example 30 minutes and can preferably be ended automatically as soon as the detection system can be classified as being in order, for example by way of a self-diagnosis procedure. Calibration of the measuring arrangements for detecting the respiratory gas pressure and the respiratory gas flow is preferably already effected at the breathing mask arrangement applied to the patient.

After the conclusion of the calibration mode initiation of the titration mode is implemented by the pressure control concept. In the context of that titration mode the respiratory gas pressure can be altered stepwise over a plurality of successive titration sequences, as was described hereinbefore by way of example with reference to FIGS. 1a to 1d. In the context of the titration mode the instantaneous configuration of the respiratory gas flow is analysed, using predetermined evaluation criteria. By applying those evaluation criteria, it is possible to generate evaluation features for the individual titration sequences and in particular for the pressure stages which are operated in that context. Those evaluation features can be stored in a data field. Indicative evaluation results can be generated, in respect of any set of symptoms that may be present, by combinational processing of the evaluation features ascertained.

Upon generation of the evaluation features, breathing disorders are preferably described by an analysis of the respiratory gas flow and the respiratory gas pressure and preferably also with the incorporation of further polysomnographic parameters such as the blood oxygen saturation content, the position of the body of the patient and EEG, ECG and/or EOG-signals.

Configurational information is ascertained on the basis of the ascertained evaluation features and the evaluation results derived therefrom, a therapy mode being implemented subsequently to the titration mode, in accordance with the configurational information.

That therapy mode follows the titration mode described hereinbefore. In the context of the therapy mode, respiration of the patient can be further monitored in particular by evaluation of the respiratory gas flow signal and the respiratory gas pressure signal. On the basis of the monitoring results, it is then possible to check the plausibility of the settings ascertained in the course of the titration mode. It is further possible also to describe the therapy quality, by evaluation of the measurement signals ascertained in the context of the therapy mode.

The titration mode can be carried out in particular in such a way that it ascertains a CPAP-pressure which is required for a CPAP-therapy and which is still validated following the titration mode. In that situation the therapy mode can be carried out in such a way that, by means of a pressure control device, the respiratory gas pressure applied to the patient is successively increased in accordance with a given pressure control concept over a plurality of titration sequences. The increase in pressure can take place in accordance with a rigidly predetermined time pattern or also on the basis of continuous analysis of the signals indicative in respect of the breathing of the patient.

It is also possible for the respiratory gas pressure applied to the patient to be reduced from a high pressure level at which no breathing disorders are expected to occur, to a predetermined minimum level, over successive consecutive titration sequences. It is also possible for the pressure control concepts described hereinbefore to be used in combination. Thus for example the pressure can be raised successively over a plurality of titration sequences to a plausible maximum level (FIG. 1a) and then lowered again over a plurality of titration sequences to the initial pressure level (FIG. 1c). It is also possible for the pressure control concepts shown in FIGS. 1a, 1b, 1c and 1d to be combined.

In the titration mode preferably the measurement signals detected therein are evaluated in regard to any indications contained therein, in respect of disturbed breathing. The nature of the breathing disorder and possibly the degree thereof can be deposited as evaluation features, preferably in association with the respiratory gas pressure which is set in that situation, in a characteristic field. The entries in that characteristic field can be combinationally evaluated simultaneously or also in the context of a subsequent evaluation procedure. On the basis of the evaluation operations which are all carried out it is then possible to establish indicative indices, in respect of a set of symptoms which are possibly present, and a therapy pressure which is possibly required.

The titration algorithm is preferably distinguished by the following features:

The process of the titration operation is performed in accordance with a standardised pressure control concept.

Detection of breathing disorders is effected by applying standardised evaluation criteria and is thus reproducible.

Detection of breathing disorders can be set selectively in accordance with various medical standards.

Detection of breathing disorders is preferably effected from the signals in respect of volume flow, pressure, oxygen saturation, body position, EOG and EEG.

An effective therapy pressure which is possibly needed by the patient is obtained from analysis of the recorded measurement signals.

The titration algorithm is preferably embedded in a calibration mode and a therapy or validation mode.

The investigation process is preferably such that the titration mode preferably extends over the first half of an investigation night and in the remaining sleeping period of the patient, the feed of the respiration gas is already implemented under the therapy conditions ascertained in the context of the therapy mode.

The change from the titration mode to the therapy or validation mode can be effected under program control, having regard to a plurality of switching-over criteria. The program execution can be determined manually, semi-automatically or also fully automatically.

In the context of the titration mode, checking of the effective therapy pressure can be effected by the pressure being reduced in a defined manner for a given interval and/or a given number of breaths. The drop in pressure can be effected by a step function or phase-wise with a return to a reference pressure level.

FIG. 3 shows an arrangement for investigating a patient 30 with sleep-related breathing disorders. The patient 30 wears a nasally applied breathing mask 31. By way of that breathing mask 31, it is possible to supply the patient 30 with ambient air at a pressure level which at least phase-wise is above the ambient pressure. The respiratory gas flow is effected by way of a flexible respiratory gas conduit 32 which is coupled by way of a pneumotachagraph 33 to the patient's own CPAP-apparatus 34.

The CPAP-apparatus is provided with an air humidifier 35 and an internal pressure regulating device. The internal pressure regulating device has a pressure measuring sensor which is acted upon in per se known manner by way of a pressure measuring hose 36. The pressure regulating device can be so configured that it interprets the pressure applied at the pressure measuring hose as the actual pressure and regulates the speed of rotation of a blower of the CPAP-apparatus, in dependence on a set reference pressure.

In the illustrated arrangement, the pressure measuring hose 36 is connected to a pressure module 37, by way of which defined pressures can be applied to the pressure measuring hose 36 by means of an auxiliary pressure source 38. By means of the pressure module 33, it is further also possible to couple the pressure measuring hose 36 switchably to a pressure measuring hose portion 36a which leads to the breathing mask. The use of the pressure module 36 and the auxiliary pressure source makes it possible for the speed of rotation of the blower of the CPAP-apparatus 34 to be controlled without intervening in the regulating system which is internal to the apparatus, and thus to adjust the pressure to the respectively desired titration sequence pressure level. As an alternative thereto, it is also possible, when the interface of the CPAP-apparatus is available, to connect same to the titration control unit 40 by way of a data line 39. If the CPAP-apparatus has a sufficiently precise gas flow measuring device and gas pressure measuring device, the corresponding measurement signals can be obtained directly by way of the CPAP-apparatus, eliminating the components 33, 37 and 38.

Processing and measurement data acquisition can be implemented, in accordance with the pressure control concepts described hereinbefore, by way of the titration control unit 40.

The acquired measurement data can be continuously evaluated by program-implemented evaluation procedures. The evaluation results which are preferably continuously ascertained and possibly continuously improved and in particular assumed suitable operating settings for the CPAP-apparatus can be displayed possibly in conjunction with particularly relevant breathing patterns on a display device 41. By way of an input device 42, for example in the form of a keyboard and/or mouse, it is possible to influence the progress of signal titration and measurement value acquisition.

After the titration mode has been implemented the illustrated arrangement can be operated at settings which were ascertained in the context of the titration mode. The quality of the respiratory gas pressure setting can be described and displayed in particular by characteristic values.

Further details in particular in regard to classification and automatic assessment of breathing in the individual titration sequences will be apparent from the description hereinafter.

The breath 1 shown in FIG. 4a in regard to the configuration in respect of time of the respiratory gas flow includes an inspiration phase I and an expiration phase E. Ascertaining the respiration phase boundary G between the inspiration phase and the expiration phase is effected by superimposed evaluation of a plurality of curve discussion criteria, in particular also having regard to the instantaneously prevailing respiration pattern and the extreme values of the respiratory gas flow, and the pattern of the ascertained breath volume and having regard to the respiration phase periods of preceding breaths. The configuration of the respiratory gas flow shown in FIG. 4a describes the respiratory gas flow pattern in an undisturbed breath.

Breath evaluation can be effected on the basis of the conditions in respect of time, for example the inspiration and expiration time relative to each other or relative to other properties, for example the overall breath length. In accordance with a particularly preferred embodiment of the invention the quotient of the inspiration time and the overall breath length is calculated in order to detect changes in breathing.

FIG. 4b shows the configuration of the respiratory gas flow over a longer time window. As can be seen from this representation the individual breaths vary in particular in terms of the minima/maxima which occur in this situation. The horizontal line 2 plotted in this view clearly marks the maximum respiratory gas flow which occurs with the highest probability, considered statistically, for inspiration phases. In addition a statistical analysis of the inspiration, expiration and overall breath times over a plurality of breaths (preferably 10 breaths) can be effected.

FIG. 4c shows the pattern in respect of time of a signal which is indicative in respect of the respiratory gas pressure, that signal having oscillation sequences 3a, 3b, 3c, 3d and 3e which are caused by snoring. The pressure fluctuations caused by snoring can be detected by way of a pressure detecting device in the proximity of the patient, for example a respiratory gas pressure measuring hose. It is also possible for pressure fluctuations of that kind to be detected by way of microphone devices or also on the basis of the power draw of a respiratory gas delivery device.

FIG. 4d shows the pattern in respect of time of the respiratory gas flow for a plurality of breaths 1 interrupted by a respiration stoppage period 5. The respiration stoppage period 5 detected on the basis of the respiratory gas flow is of a time duration which exceeds a predetermined limit value of for example 10 seconds and is thus classified as an apnoea phase. Both the breaths detected in that representation prior to the respiration stoppage period 5 and the subsequent breaths exhibit flow limitation features which are plotted in association with each breath.

FIG. 5 shows the pattern in respect of time of the respiratory gas flow with a hypopnoea phase 6 included therein. The hypopnoea phase 6 is deemed to be present if three breaths 1 which are classified as normal are followed by at least two but a maximum of three breaths, the inspiratory difference volume thereof exceeding a predetermined limit value, in comparison with the three preceding breaths.

FIG. 6 shows the pattern in respect of time of the respiratory gas flow for a plurality of breaths, wherein the first four breaths 1 visible here exhibit flow limitation features. Those flow limitation features can be recognised in the illustrated configuration of the respiratory gas flow by plateaux 7 formed therein and by a plurality of local maxima 8. In the case of the illustrated breaths the flow limitation features respectively occur in the inspiration phase of the respective breath 1. The first four breaths 1 illustrated here are followed by three breaths oh, which are in part still flow-limited, which are to be associated with a hypopnoea phase and in part also still exhibit flow limitation features.

FIG. 7 shows the pattern of the respiratory gas flow in a respiration period which is classified as stable. The respiratory gas flow, the respiratory rate, the amplitude and the pattern of the respiratory gas flow are regular within a predetermined range which can be defined as a time range or also by a number of breaths. In the configuration of the respiratory gas flow illustrated here the respiration stability moves above a respiration stability limit value of 0.86. In addition a statistical analysis of the inspiration time/expiration time and overall breath time can be effected over a plurality of breaths (preferably 10 breaths). No respiratory disturbances (OSA) occur during the phase of stable respiration, which is illustrated here.

FIG. 8 shows the pattern in respect of time of the respiratory gas flow for a plurality of breaths, wherein the respiratory flow is irregular during the illustrated time section and respiratory disturbances (OSA) occur in regard to some breaths. In addition a statistical analysis of the inspiration time/expiration time and overall breath time can be effected over a plurality of breaths (preferably 10 breaths). In the embodiment illustrated here the respiration stability index is below the limit value of 0.911.

FIG. 9 shows the pattern in respect of time of the respiratory gas flow in conjunction with a respiratory gas pressure signal. The respiratory gas pressure signal contains phase-wise high-frequency oscillations which in the present example can be associated with inspiratory snoring.

FIG. 10 shows the pattern in respect of time of the respiratory gas flow for a plurality of breaths, wherein respiration is phase-wise irregular and from the moment in time T1 there is a disturbance caused for example by mask leaks or by the mouth being open. From the moment T1 a predetermined limit value is exceeded, which limit value can be assessed as an indication of a system disturbance due to mouth breathing or mask leaks.

Generation in accordance with the invention of an evaluation result which is indicative in respect of the physiological state of a patient can be used to control the respiratory gas pressure in the context of over-pressure artificial respiration. A situation of use of that kind is described hereinafter with reference to FIGS. 11 through 21. The feed of the respiratory gas to the patient is effected using a nasally applied respiratory mask which is connected by way of a respiratory gas hose to a respiratory gas source which provides respiratory gas at a variably adjustable pressure level. That respiratory gas supply arrangement includes a pressure detecting device for generating a signal which is indicative in respect of the respiratory gas pressure and a respiratory gas flow detecting device for detecting a signal which is indicative in respect of the respiratory gas flow. The signal which is indicative in respect of the respiratory gas flow is analysed by an evaluation device which generates evaluation features, using predetermined evaluation systems. Those evaluation features are considered in interlinked relationship and, when predetermined interlinking criteria are satisfied, they result in changes in the respiratory gas pressure or details for classification of the patient.

In the pattern illustrated in FIG. 11 of the signal which is indicative in respect of the respiratory gas flow, after the tenth breath illustrated here there is a first respiratory disturbance classified as an apnoea phase, the duration of which is about 15 seconds. That apnoea phase is followed by a series of breaths which in part have flow limitation features. Those partly flow-limited breaths are followed by a second phase of disturbed respiration, which is classified as an apnoea phase and which extends over a period of also 15 seconds. That second apnoea phase is followed by a number of (here six) breaths which in part have flow limitation-indicative features. That breath sequence is followed by a phase of disturbed respiration, which is here classified as a third apnoea phase. Following that third apnoea phase, there are three breaths, the respiration volume of which exceeds an adaptively adapted limit value and they are thus associated with a hypopnoea phase. Due to the specified occurrence of the above-mentioned three apnoea phases, the spacing in respect of time of the apnoea phases relative to each other and having regard to the hypopnoea phase which follows the third apnoea phase, an interlinking criterion is satisfied, and thus an evaluation result is generated, which assesses the respiratory gas pressure adjusted hitherto as being excessively low and causes an increase in pressure by a pressure level of 2 mbars. The breaths occurring after an increase in the respiratory gas pressure to a pressure of 11 mbars are further analysed in respect of features contained therein and considered in interlinked relationship over a larger time window.

FIG. 12 shows the pattern in respect of time of the respiratory gas flow, wherein evaluation of the detected respiratory flow signals detects a flow-limited respiration and at predetermined time intervals successively results in an increase in the respiratory gas pressure until respiration which is to be classified as normal occurs.

The configuration shown in FIG. 13 for the signal indicative in respect of the respiratory gas flow provides that a first breath sequence is classified as a sequence of stable respiration, wherein the state of stable respiration, which lasts over a predetermined period of time, causes a reduction in the respiratory gas pressure. The signals which are generated at that reduced respiratory gas pressure and which are indicative in respect of the respiratory gas pressure permit conclusions to be drawn about a partly flow-limited respiration.

Having regard to the flow limitation features which can be detected in the breaths, the respiratory gas pressure is increased again. The new respiratory gas pressure level however is at least temporarily below the pressure level at which a stable respiration was previously detected.

The pattern shown in FIG. 14 of the signal indicative in respect of the respiratory gas flow exhibits a plurality of apnoea phases, in part with subsequent hypopnoea phases. The position in respect of time of the apnoea and the hypopnoea phases relative to each other leads to an evaluation result which classifies the prevailing respiratory gas pressure as inadequate and causes an increase in the respiratory gas pressure.

The pattern illustrated in FIG. 15 of the signal indicative in respect of the respiratory gas flow shows three breath sequences which can be classified as hypopnoea sequences. The position in respect of time of the hypopnoea sequences relative to each other leads to an evaluation result which classifies the prevailing respiratory gas pressure as inadequate and causes an increase in the respiratory gas pressure. After the increase in respiratory gas pressure the configuration of the signal which is indicative in respect of the respiratory gas flow reveals a respiration which is to be classified as normal.

FIG. 16 shows a sequence of the signal which is indicative in respect of the respiratory gas flow and which shows flow limitation features for the individual breaths, wherein oscillations which can be classified as inspiratory snoring occur at the same time as the occurrence of flow limitation features in the breaths in the respiratory gas pressure signal.

The flow limitation features which occur in the individual breaths, in conjunction with the oscillations detected in the respiratory gas pressure signal, lead to an evaluation result which describes the prevailing respiratory gas pressure as inadequate and consequently causes an increase in the respiratory gas pressure.

The breaths detected after the increase in respiratory gas pressure are classified as breaths of normal respiration.

As soon as the state of normal respiration persists over a predetermined period of time, as shown in FIG. 17, the respiratory gas pressure can be lowered by for example 2 mbars. That reduced respiratory gas pressure level is maintained until even thereat no flow limitation features can be detected in the individual breaths. If at that pressure level a respiration which is to be classified as normal occurs over a predetermined period of time, the respiratory gas pressure can be further reduced.

After that phase of normal respiration the respiratory gas pressure can be further reduced, as shown in FIG. 18. If flow limitation features occur at that further reduced respiratory gas pressure, in the individual breaths detected, the respiratory gas pressure can be increased again, on the basis of interlinked consideration of the breath features ascertained for the individual breaths.

FIG. 18 further shows the configuration of the signal which is indicative in respect of the respiratory gas flow, in the case of a system disturbance caused for example by mask leakage. The respiratory gas pressure drop which is detected in that situation and the rise in the respiratory gas flow, which occurs at the same time therewith, leads to the generation of an evaluation result which assesses the instantaneous system state as disturbed. The system according to the invention is adapted in such a way that, in the case of a disturbance classified as mask leakage, the delivery output of the respiratory gas source is matched in such a way that the respiratory gas pressure prevailing until the occurrence of the disturbance remains substantially maintained.

As can be seen from the view in FIG. 19 a system disturbance which has occurred for example due to temporary displacement of a respiration mask and which is classified as mask leakage can be removed again for example after changing the head position of the patient and respiration can be continued under the respiratory gas pressure which was also maintained during the system disturbance. On the basis of the signal which is indicative of the respiratory gas flow, it is also possible to ascertain whether the situation involves mouth breathing, as can be seen from FIG. 20.

FIG. 21 shows the configuration in respect of time of a signal S which is indicative in respect of the respiratory gas flow. That signal is recorded for example as a so-called raw data signal by a pressure sensor connected to a dynamic pressure measuring location, with a sampling frequency of between for example 10 and 500 Hz. The raw data signal S can be recorded by way of an approximation system 20 using approximation procedures implemented therein, for example series developments in the form of fast Fourier analysis, a (for example) MP3 compression, Laplace series development, binomial series development, correlation series development and so forth in compressed form.

The possibly compressed raw data of the signal S can be recorded within a data sequence D.

In the data sequence D, evaluation features M can further be generated, using a plurality of evaluation systems 21, which features for example describe certain properties of breaths or periods of time.

On the basis of the possibly compressed raw data of the signal S and/or the evaluation features M, at least one evaluation result is generated in the context of a result-generation step, by the evaluation features M being subjected to interlinked consideration.

In the situation involving use of the system according to the invention for setting a respiratory gas pressure, one of the evaluation results can be a signal which for example specifies the instantaneous respiratory gas pressure as suitable, too low or too high. A change value which is possibly required in respect of the respiratory gas pressure can be ascertained as a further evaluation result. Regulating parameters for setting and synchronising the respiratory gas pressure in a bi-level pressure control can also be ascertained as evaluation results.

The interlinking consideration of the evaluation features M is preferably effected with the incorporation of Boolean operations, wherein the Boolean variables A₁, A₂, B₁, . . . E₂ . . . are generated from individual evaluation features M and/or by combinational evaluation of the evaluation features M, for example evaluation feature groups a₁, a₂, b₁, c₂, . . . . The evaluation results can be the outcome of a plurality of OR-interlinked operational systems.

On the basis of the evaluation results it is possible to select raw data sets or evaluation feature sets which are used for the generation of desired information such as for example a pressure change value and typification indices (FLI, snoring index, . . . ).

The approximation system 20, the evaluation systems 21 and the systems for interlinking consideration of the evaluation features M and the preparatory generation of Boolean variables are preferably afforded by a computing device which is configured by means of a program data set.

The evaluation results can be generated in the framework of a data post-processing procedure or used in real time—or in sufficiently close time relationship—in setting a respiratory gas pressure or configuring a pressure control system.

The evaluation results can be made available to a pressure control algorithm, which is preferably such that, in a respiratory gas pressure regulation procedure, it affords at least two pressure regulating modes which differ in terms of their reaction behaviour. Thus it is possible to operate a respiratory gas pressure control system in a base mode in which given events or a sum of events causes an increase in the respiratory gas pressure.

In the context of a sensitive mode it is possible for pressure control to be effected in such a way that it reacts to possibly detected events with a minor delay. That sensitive mode can be set in particular when the respiratory gas pressure was reduced for example after a phase of stable respiration (RS≧0.911).

In accordance with the base mode it is preferably provided that an increase in pressure is caused to occur when two large or three small apnoeas occur and the respiratory gas pressure is less than 14 mbars or a respiration stoppage is detected, which exceeds a predetermined time duration of for example 2 minutes. In that case a pressure increase by two mbars can be caused.

In the base mode a pressure increase by 1 mbar can be caused preferably when three hypopnoea sequences are detected in a predetermined time succession. Pressure increases by a pressure level of 1 mbar are preferably caused when, with a respiration stability index ≧0.911, flow limitations occur at A out of B or also C out of D breaths.

The base mode is further preferably adapted in such a way that it causes a reduction in pressure when stable respiration occurs with a respiration stability index RS≧0.911 over a period of time of at least 9 minutes. In that case a reduction in pressure by preferably 2 mbars is caused. In the context of the base mode, a change in pressure is suppressed in particular when the respiration stability index≦0.911 and the limitation phenomena which are detected in that case, in the individual breaths, do not exceed a predetermined severity criterion.

In the context of the sensitive mode an increase in the respiratory gas pressure by for example 2 mbars is caused when two large or three small apnoeas occur and the respiratory gas pressure is ≦14 mbars. When three hypopnoea sequences occur an increase in the respiratory gas pressure by 1 mbar occurs.

Upon the occurrence of flow limitation features in the breaths being investigated, an increase in the respiratory gas pressure by 1 mbar is caused when four out of B breaths have flow limitation features and the respiration stability index is ≧0.87. A pressure increase by 1 mbar is also caused when C out of D breaths have flow limitation features and the respiration stability index is ≦0.911. If D out of B breaths have flow limitation features and if the respiration stability index is below a value of 0.911 an increase in the respiratory gas pressure by 1 mbar also occurs in the sensitive mode.

A reduction in the respiratory gas pressure already occurs in the sensitive mode when stable respiration occurs over a period of 3 minutes and the respiration stability index is ≧0.911. In that case the respiratory gas pressure can be reduced by for example 2 mbars.

Similarly as also in the above-mentioned base mode, no change in pressure is caused in the sensitive mode if respiration is classified as unstable and if flow limitation features can be detected in the individual breaths with a respiration stability index≦0.911.

Both in the normal mode and also in the sensitive mode it is preferably provided that events such as swallowing, coughing, mouth respiration, in particular expiratory mouth respiration, arousals and talking, do not cause any change in respiratory gas pressure at least when the respiratory gas pressure is below a limit value of for example 14 mbars.

Interlinking consideration can for example result in changes in pressure. It can also result in the calculation of patient-typical indices by a procedure whereby those relevant measurement data are selected by same, which are relevant in relation to the respective index and were ascertained in a patient study which has a high capacity for providing information.

1. A method of generating an evaluation result which is specific in respect of the physiological state of a person, on the basis of measurement signals which are in a relationship with the respiration of the person, wherein evaluation features are generated from said measurement signals, using a plurality of evaluation systems, and at least one evaluation result is generated in the context of a result-generation step based thereon, by the evaluation features being subjected to interlinking consideration, and the measurement signals are detected in titration sequences which are different in respect of the respiratory gas pressure level applied to the patient and the generation of at least a part of the evaluation features or the evaluation result is effected having regard to the respective titration sequence pressure.

2. A method as set forth in claim 1 characterised in that the titration sequence pressure is substantially constant within a titration sequence.

3. A method as set forth in claim 1 characterised in that the titration sequence pressure follows a pressure control concept within a titration sequence.

4. A method as set forth in claim 1 characterised in that the length in respect of time of the titration sequence is determined by sequence length criteria.

5. A method as set forth in claim 4 characterised in that the sequence length criteria include criteria of a minimum time duration.

6. A method as set forth in claim 4 characterised in that the sequence length criteria include criteria in respect of a minimum respiration number.

7. A method as set forth in claim 4 characterised in that the sequence length criteria include obstruction indicators.

8. A method as set forth in claim 4 characterised in that the sequence length criteria include a forward-switching criterion.

9. A method as set forth in claim 1 characterised in that the pressure control within a titration sequence is matched to the detection of given indicators, wherein included therein are indicators for central breathing disorders and/or obstructive breathing disorders and/or patient-specific breathing patterns.

10. A method as set forth in claim 9 characterised in that apnoea indicators are among the indicators.

11. A method as set forth in claim 9 characterised in that hypopnoea indicators are among the indicators.

12. A method as set forth in claim 9 characterised in that flow limitation indicators are among the indicators.

13. A method as set forth in claim 1 characterised in that actuation of the titration sequences is effected in accordance with a sequence control concept.

14. A method as set forth in claim 13 characterised in that the sequence control concept includes at least one period of successively rising pressure stages or that the sequence control concept includes at least one period of successively falling pressure stages.

15. A method as set forth in claim 13 characterised in that the sequence control concept provides a plurality of titration sequences with different titration sequence pressures, wherein in the context of actuation of said titration sequence pressures intermediate phase pressures are actuated, in which the respiratory gas pressure level is at a level which is higher than the titration sequence pressure of a preceding titration sequence and a subsequent titration sequence.

16. A method as set forth in claim 15 characterised in that the intermediate phase pressures are each at the same respective pressure level.

17. A method as set forth in claim 15 characterised in that the intermediate phase pressures are at an expected suitable therapy pressure.

18. A method as set forth in claim 13 characterised in that the sequence control concept extends over a titration period and a validation period follows the titration period.

19. A method as set forth in claim 18 characterised in that adaptation or plausibility checking of the evaluation results is effected during a validation period.

20. A method as set forth in claim 18 characterised in that in the context of the validation period suitably testing of a patient-specific pressure control configuration is effected.

21. A method as set forth in claim 1 characterised in that a feature contribution which is predominantly contained in the evaluation features is generated within a generation time window which is smaller than an interlinking time window provided for the interlinking consideration.

22. A method as set forth in claim 1 characterised in that physiological typification of the patient in relation to obstructive, central and/or hybrid breathing disorders is effected on the basis of the interlinking consideration.

23. A method as set forth in claim 1 characterised in that a configuration data network is generated on the basis of the interlinking consideration, for the configuration of the respiratory gas pressure regulation of a respiratory gas feed device.

24. A method as set forth in claim 1 characterised in that the evaluation features are generated on the basis of breath stability criteria.

25. A method as set forth in claim 1 characterised in that the evaluation features are generated on the basis of statistical evaluation procedures.

26. A method as set forth in claim 1 characterised in that the evaluation features are generated as a feature field.

27. A method as set forth in claim 1 characterised in that normal respiration phase lengths and/or normal respiration-characteristic features and/or features for regular or irregular respiration phase lengths and/or regular and/or irregular features, characteristic evaluation features, are generated as the evaluation feature.

28. A method as set forth in claim 1 characterised in that flow limitation phase lengths and/or flow limitation-characteristic features or data sets and/or features for obstructive breathing disorders and/or obstruction-characteristic features are generated as the evaluation features.

29. A method as set forth in claim 1 characterised in that apnoea-characteristic features or data sets are generated as evaluation features.

30. A method as set forth in claim 1 characterised in that snoring phase lengths and/or snoring phase-characteristic features are generated as evaluation features.

31. A method as set forth in claim 1 characterised in that features which are indicative in respect of the occurrence of central and/or hybrid breathing disorders or in respect of the ratio of the proportion or the duration of central to hybrid or central to obstructive breathing disorders are generated as evaluation features.

32. A method as set forth in claim 1 characterised in that features for Cheyne-Stokes phase lengths or Cheyne-Stokes characteristic features or data sets are generated as evaluation features.

33. A method as set forth in claim 1 characterised in that features in respect of periodic processes, for example the phase length of periodic processes, are generated as evaluation features.

34. A method as set forth in claim 1 characterised in that features for the hypoventilation phase lengths or hyperventilation-characteristic features or data sets are generated as evaluation features.

35. A method as set forth in claim 1 characterised in that features in respect of breath-specific times, for example features in respect of the inspiration time, the expiration time and the overall cycle, are ascertained as evaluation features.

36. A method as set forth claim 1 characterised in that features in respect of the maximum respiration volume flow of inspiration and expiration are generated as evaluation features.

37. A method as set forth in claim 1 characterised in that mouth breathing-indicative features of inspiration and/or expiration are ascertained as evaluation features.

38. A method as set forth in claim 1 characterised in that lung draw volume-indicative features or data sets are generated as evaluation features.

39. A method as set forth in claim 1 characterised in that body position-indicative features or data sets are generated as evaluation features.

40. A method as set forth in claim 1 characterised in that sleep phase-characteristic features or data sets are generated as the evaluation feature.

41. A method as set forth in claim 1 characterised in that titration-characteristic features, for example titration mode phases, or data sets or titration measurement values are used as the evaluation feature.

42. A method as set forth in claim 1 characterised in that special intervals of the titration sequences or data sets are generated or recorded as the evaluation feature.

43. A method as set forth in claim 1 characterised in that features in respect of the proportion or degree of leakage are generated as the evaluation feature.

44. A method as set forth in claim 1 characterised in that leakage times are stored as the evaluation feature.

45. A method as set forth in claim 1 characterised in that the titration differential pressure is stored as the evaluation feature.

46. A method as set forth in claim 1 characterised in that the initial and/or end titration pressure is ascertained and/or recorded as the evaluation feature.

47. A method as set forth in claim 1 characterised in that the titration pressure pattern is recorded as the evaluation feature.

48. A method as set forth in claim 1 characterised in that an inspiration volume flow/pressure diagram in dependence on detected breathing disorders is generated as the evaluation feature.

49. A method as set forth in claim 1 characterised in that generated evaluation features are stored with association in respect of their position in respect of time in the measurement signal acquisition period.

50. A method as set forth in claim 1 characterised in that a flow limitation index is generated in the context of the interlinking consideration.

51. A method as set forth in claim 1 characterised in that an apnoea/hypopnoea index is generated in the context of the interlinking consideration.

52. A method as set forth in claim 1 characterised in that a snoring index is generated in the context of the interlinking consideration.

53. A method as set forth in claim 1 characterised in that a mouth breathing/nasal breathing index is generated in the context of the interlinking consideration.

54. A method as set forth in claim 1 characterised in that a sleep time index is generated in the context of the interlinking consideration.

55. A method as set forth in claim 1 characterised in that a sleep phase index is generated in the context of the interlinking consideration.

56. A method as set forth in claim 1 characterised in that a periodic respiration index is generated in the context of the interlinking consideration.

57. A method as set forth in claim 1 characterised in that a respiration volume index is generated in the context of the interlinking consideration.

58. A method as set forth in claim 1 characterised in that in the context of the interlinking consideration the evaluation features are taken into consideration with a weighting which is determined for the respective interlinking.

59. A method as set forth in claim 1 characterised in that the evaluation features are generated on the basis of a v-measurement.

60. A method as set forth in claim 1 characterised in that at least a part of the evaluation features is generated having regard to the first and/or the second derivative of the configuration in respect of time of the respiratory gas flow.

61. A method as set forth in claim 1 characterised in that in the context of detecting the v-signal the pressure of the respiratory gas which flows to the patient corresponds to the ambient pressure.

62. A method as set forth in claim 1 characterised in that in the context of detecting the v-signal the respiration gas pressure is set to a pressure level which differs from the ambient pressure.

63. Apparatus for generating an evaluation result which is specific in respect of the physiological state of a breathing person, on the basis of measurement signals which are related to the respiration of the person, comprising a measurement signal input device and a computing device for providing a plurality of evaluation systems, wherein the computing device is configured in such a way that it generates evaluation features from said measurement signals by the evaluation systems and said evaluation features are subjected in the context of a result-generation step based thereon to interlinking consideration and an output signal or an output data set which contains the evaluation result is generated on the basis of the interlinking consideration, and the measurement signals are detected in titration sequences which are different in respect of the respiratory gas pressure level applied to the patient and the generation of at least a part of the evaluation features or the evaluation result is effected having regard to the respective titration sequence pressure.

64. A method of patient-specific configuration of a CPAP-apparatus in which in the context of a titration period in respect of the physiological state of a person specific evaluation results are obtained on the basis of measurement signals which are in a relationship with the respiration of the person, wherein evaluation features are generated from said measurement signals, using a plurality of evaluation procedures, and said patient-specific setting of the CPAP-apparatus is effected in dependence on said evaluation features, and following the titration period operation of the CPAP-apparatus is effected under therapy conditions which are ascertained as suitable, wherein in the context of the titration mode control of the respiratory gas pressure is effected in accordance with a pressure regulating concept which under program control causes the setting of different respiration gas pressure levels in such a way that the measurement signals are detected in titration sequences which are different in respect of the respiratory gas pressure level applied to the patient, wherein the generation of at least a part of the evaluation features is effected having regard to the respective titration sequence pressure.

65. A method as set forth in claim 64 characterised in that the titration period extends over the first 30% of the sleep period of the patient.

66. A method as set forth in claim 65 characterised in that the titration period and the subsequent validation period are executed in the course of a stay on the part of the patient in a sleep laboratory.

67. A method as set forth in claim 64 characterised in that the titration period is implemented using the CPAP-apparatus provided for the therapy.

68. A method as set forth in claim 67 characterised in that the therapy apparatus can be coupled to a control unit which at least during the titration period causes operation of the apparatus in accordance with a pressure control concept for actuating a plurality of titration sequence pressure levels.

69. A method as set forth in claim 64 characterised in that a patient-specific effective therapy pressure is ascertained by the titration method.

70. A method as set forth in claim 64 characterised in that assessment bases for a prognosis of breathing-related illnesses are afforded by the titration method.

71. A method as set forth in claim 64 characterised in that evaluation results are generated by the titration method, which results permit classification or assessment of a patient in respect of obstructive, central and/or hybrid breathing disorders or the provision of a therapy recommendation.

72. A method as set forth in claim 64 characterised in that a standardised and protocolled diagnostic procedure is operated by the titration method.

73. A method as set forth in claim 64 characterised in that the evaluation features are convertible into various medical defined standard assessments.

74. A method as set forth in claim 64 characterised in that assessment bases are generated (prior to the actual illness) diagnosed or prognosticated.
